(19) United States
(12) Patent Application Publication
(10) Pub. No.: US 2026/0268493 A1
(43) Pub. Date: Sep. 10, 2026

(54) TECHNIQUES FOR MEASURING ATYPICAL NEURODEVELOPMENT IN NEONATES BASED ON SHORT VIDEO

(71) Applicant: Rutgers, The State University of New Jersey, New Brunswick, NJ (US)

(72) Inventor: Elizabeth B. TORRES, Piscataway, NJ (US)

(21) Appl. No.: 18/869,437

(22) PCT Filed: Jun. 1, 2023
(86) PCT No.: PCT/US23/67728

§ 371 Date: Nov. 26, 2024

Related U.S. Application Data
(60) Provisional application No. 63/347,608, filed on Jun. 1, 2022.

(51) Int. Cl. G06T 7/00; G06T 7/20; G16H 30/40; G16H 50/20; G16H 50/70

(52) U.S. Cl. CPC G06T 7/0016; G06T 7/20; G16H 30/40; G16H 50/20; G16H 50/70; G06T 2207/10016; G06T 2207/20084

(57) ABSTRACT

Techniques for determining a neurodevelopmental condition of a subject includes recording video data of a subject. A micromovements profile of each of one or more anatomical locations on the subject is determined using pose algorithms on the recorded video data. A first empirical distribution of micromovement spikes is determined in each micromovement profile. A condition of the subject is determined based on a distance of the first empirical distribution from a predetermined distribution of micromovement spikes in a population of control individuals. The population of control individuals is associated with a neurodevelopmental condition.

### CROSS-REFERENCE TO THE RELATED APPLICATION

This application claims priority to PCT Application No. PCT/US23/67728, filed Jun. 1, 2023, which claims the benefit of U.S. Provisional Patent Application No. 63/347,608, filed Jun. 1, 2022, under 35 U.S.C. § 119, which is incorporated herein by reference in its entirety.

### BACKGROUND

Assessment of neurodevelopmental disorders is challenging in neonates and other preverbal children. Previous methods by the current inventors have looked at micromovements in directed versus undirected activities that are not compatible with preverbal children. Yet, an early diagnosis of atypical neurodevelopment can be useful in early treatment and intervention. An example of a widely used test for neurodevelopmental progress is the Auditory Brainstem Response (ABR) test. While sensitive to atypical development in nerves included in auditory processing, such tests have not been fully utilized for detection of other neurodevelopment disorders such as autism spectrum disorder (ASD).

### SUMMARY

Techniques are provided for using short video of neonates before or during ABR tests to detect differences in neurodevelopment and diagnose any neurodegenerative conditions, such as autism spectrum disorder (ASD).

In a first set of embodiments, a method for determining a condition of a subject includes recording video data of a subject. The method also includes determining a micromovements profile of each of one or more anatomical locations on the subject using pose algorithms on the recorded video data. The method further includes determining a first empirical distribution of micromovement spikes in each micromovement profile. Still further, the method includes determining a condition of the subject based on a distance of the first empirical distribution from a predetermined distribution of micromovement spikes in a population of control individuals.

In some embodiments of the first set, the population of control individuals includes a plurality of populations of control individuals, each population having a different predetermined distribution of micromovement spikes and each population associated with a different neurodevelopmental condition.

In some embodiments of the first set, recording video data includes recording video data of a subject before or after, or both, an auditory signal is delivered to the subject during an auditory brainstem response (ABR) test. In some of these embodiments, the predetermined distribution of micromovement spikes in the population of control individuals is associated with a predetermined distribution of micropeaks in ABR test data for the population of control individuals; and the predetermined distribution of micropeaks in ABR test data is associated with a particular neurodevelopmental condition.

In some embodiments of the first set, the plurality of populations of control individuals, includes a first population associated with normal development and different second population associated with autism spectrum disorder (ASD).

In some embodiments of the first set the first empirical distribution of micromovement spikes is an empirical distribution of a prominence or a width or an amplitude or a relative latency, or some combination, for each micromovement spike.

In some embodiments of the first set, the distance is an Earthmover's distance between distributions.

In some embodiments of the first set, the first empirical distribution is described by a plurality of distribution parameters. In some of these embodiments, the distribution parameters include mean, variance and skewness, or shape and scale of a Gamma Function, or some combination.

In other sets of embodiments, a non-transitory computer readable medium, or an apparatus, or a system is configured to perform one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example video neonate neurodevelopment test system, according to an embodiment;

FIG. 2A is a block diagram that illustrates an example pose structure for a neonate, according to an embodiment;

FIG. 2B is a diagram that illustrates an example superposition of the pose structure of FIG. 2A onto a video frame of a subject neonate, according to an embodiment;

FIG. 2C is a diagram that illustrates an example confidence matrix for the pose algorithm to detect each of 25 joints in each five-frame bin of a video clip including 600 frames (20 second clip at 29 frames per second, fps), according to an embodiment;

FIG. 2D is a diagram that illustrates an example confidence matrix for the pose algorithm to detect each of 10 joints detected with at least 90% confidence in each five-frame bin of a video clip including 600 frames (one minute clip at 10 frames per second), according to an embodiment;

FIG. 2E is a graph that illustrates example two-dimensional trajectories (X-Y plane) of four distal joints (left and right wrist and ankle) from the pose algorithm, according to an embodiment;

FIG. 2F is a graph that illustrates example one-dimensional profiles (X or Y coordinate) of four distal joints (left and right wrist and ankle) from the pose algorithm, according to an embodiment;

FIG. 3A through FIG. 3D are graphs that illustrate example speed micromovement profiles of four distal joints (left and right wrist and ankle, respectively), according to an embodiment;

FIG. 4A and FIG. 4B are graphs that illustrate example properties of micromovement spikes, such as might be found in micro movement profiles such as depicted in FIG. 3A through FIG. 3D, above), according to an embodiment;

FIG. 4C is a graph that illustrates example normalized micromovement spikes (MMS) in the micromovement profiles, according to an embodiment;

FIG. 5A is a graph that illustrates example Gamma Function parameters values for distributions of normalized MMS in the four distal joints, which detect differences in neurodevelopment, according to an embodiment;

FIG. 5B is a graph that illustrates example distributions of normalized MMS in the right ankle based on the Gamma function parameter values plotted in FIG. 5A, which detect differences in neurodevelopment, according to an embodiment;

FIG. 6 is a block diagram that illustrates example distance between distributions of micromovements for the two populations at each pair of distal joints, according to an embodiment;

FIG. 7A is a block diagram that illustrates an Auditory Brainstem Response (ABR) test setup, according to an embodiment;

FIG. 7B is a block diagram that illustrates an ABR test electrical signal recorded after each of three sounds of increasing volume, according to an embodiment;

FIG. 8A through FIG. 8D are graphs that illustrate example differences in distributions of micropeaks in ABR electrical signals between ASD and nonASD populations for all three sound volumes, according to an embodiment;

FIG. 8E through FIG. 8H are graphs that illustrate example differences in skewness ABR micropeaks compared to skewness in MMS between ASD and nonASD populations for all three sound volumes at four different ages of neonates, according to an embodiment;

FIG. 9 is a flow diagram that illustrate an example method to detect differences in neurodevelopment of a subject based on distributions of MMS, according to an embodiment;

FIG. 10A is a graph that illustrates an example growth chart presentation for showing changes in a property of a distribution with age for two different populations, according to an embodiment;

FIG. 10B is a block diagram that illustrates example temporal epochs in a neonate's development suitable for at home video capture using the method of FIG. 9, according to an embodiment;

FIG. 10C through FIG. 10E are diagrams that illustrate example results of the pose module fit to a neonate in the supine position at 6 days, 5 weeks and 17 weeks, respectively, according to an embodiment;

FIG. 10F through FIG. 10J are diagrams that illustrate example postures for the parent or other caregiver to set the neonate for video-capture, according to an embodiment

FIG. 11 is a block diagram that illustrates a computer system upon which an embodiment of the invention may be implemented;

FIG. 12 is a block diagram that illustrates a chip set upon which an embodiment of the invention may be implemented; and

FIG. 13 is a block diagram of example components of a mobile terminal (e.g., cell phone handset) for communications, which is capable of operating in the system of FIG. 1A, according to one embodiment.

### DETAILED DESCRIPTION

Techniques are described to detect or quantify differences in neurodevelopment in neonates and diagnose any neurodegenerative conditions. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of detecting Autism Spectrum Disorder (ASD) in neonates. However, the invention is not limited to this context. In other embodiments, the same or different neurodevelopmental disorders are detected or quantified in neonates, or in young children up to about 7 years of age, or some combination. A variety of neurodevelopmental disorders are expected to be detected or quantified using these methods including language related disorders, cerebral palsy and a variety of disorders incorporated by the term ASD such as Attention Deficit/Hyperactivity Disorder (ADHD), sensory disorders, and others under the Diagnostic and Statistical Manual of Mental Disorders, Fifth Edition (DSM V) criteria.

### 1. Overview

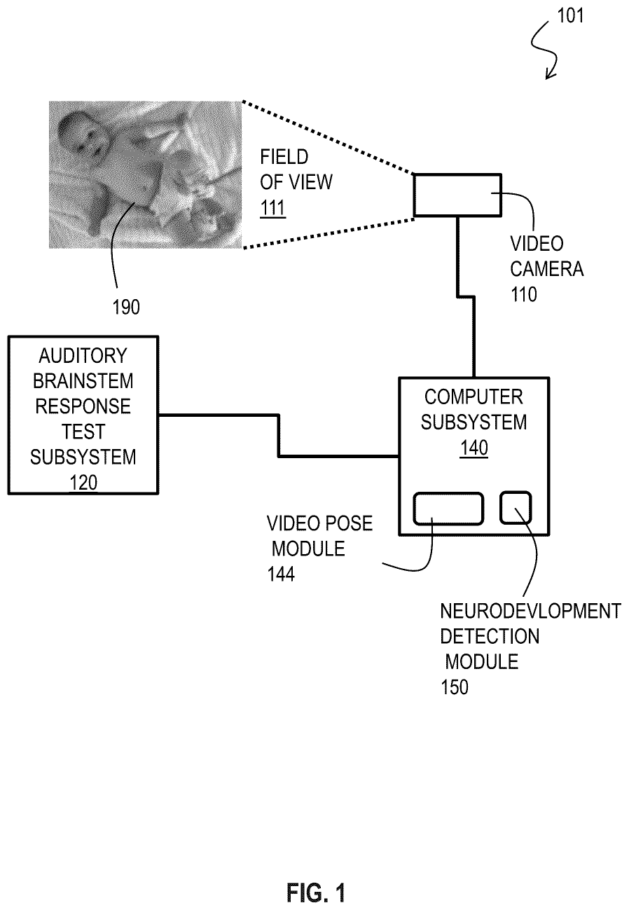

FIG. 1 is a block diagram that illustrates an example video neonate neurodevelopment test system 101, according to an embodiment. The system 101 includes a video camera 110 configured to capture and record digital video data of objects in a field of view 111, such objects including one or more subjects, such as neonate subject 190. Although neonate subject 190 is included in FIG. 1 for purposes of illustration, the subject 190 is not part of system 101. The system 101 also includes a computer subsystem 140 configured to process the digital video data. The computer subsystem 140 includes a video pose module 144 configured to detect the spatial arrangement of a variety of anatomical joints of subject 190 in each frame of the video data. The computer subsystem 140 includes a neurodevelopment detection module 150 configured to infer the neurodevelopmental condition of a subject captured in the video data based on distribution of micromovement spikes (MMS) in each of one or more joints detected by the video pose module 144, as described in more detail below. As used herein, a micromovement is motion accomplished on the time scales of a tens of microseconds to hundreds of milliseconds.

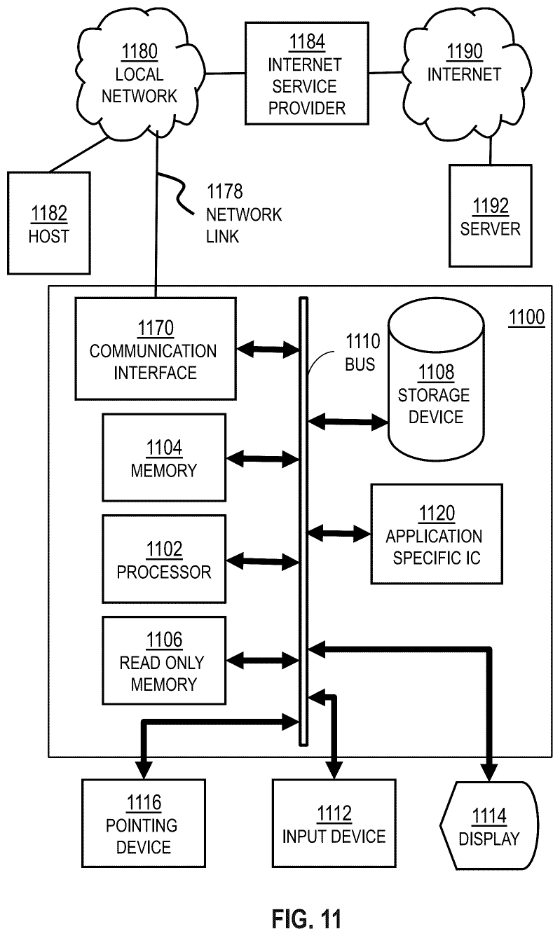

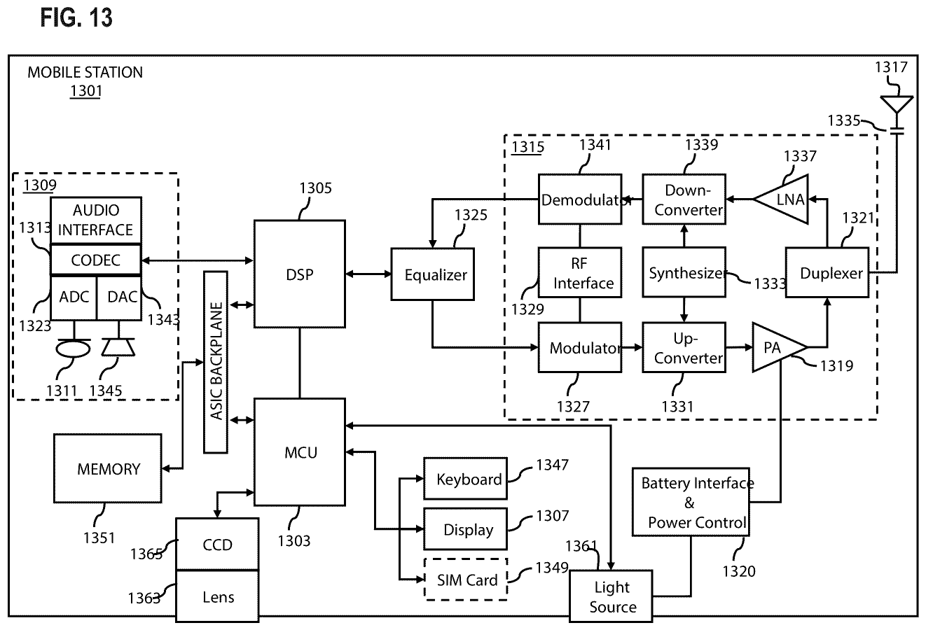

In some embodiments, the computer subsystem 140 includes one or more computer systems or hosts, such as computer system 1100 described in more detail below with reference to FIG. 11. In some embodiment the computer subsystem 140 includes one or more chip sets in one or more special devices, such as chip set 1200 described in more detail below with reference to FIG. 11. In some embodiment the computer subsystem 140 includes one or more mobile communication terminals, such as mobile terminal 1300 described in more detail below with reference to FIG. 13. In some of these embodiments, the video camera 110 is included within the mobile terminal 1300, such as in a modern smart cell phone, including lens 1363, charge coupled device 1365 and light source 1361 as described in more detail below.

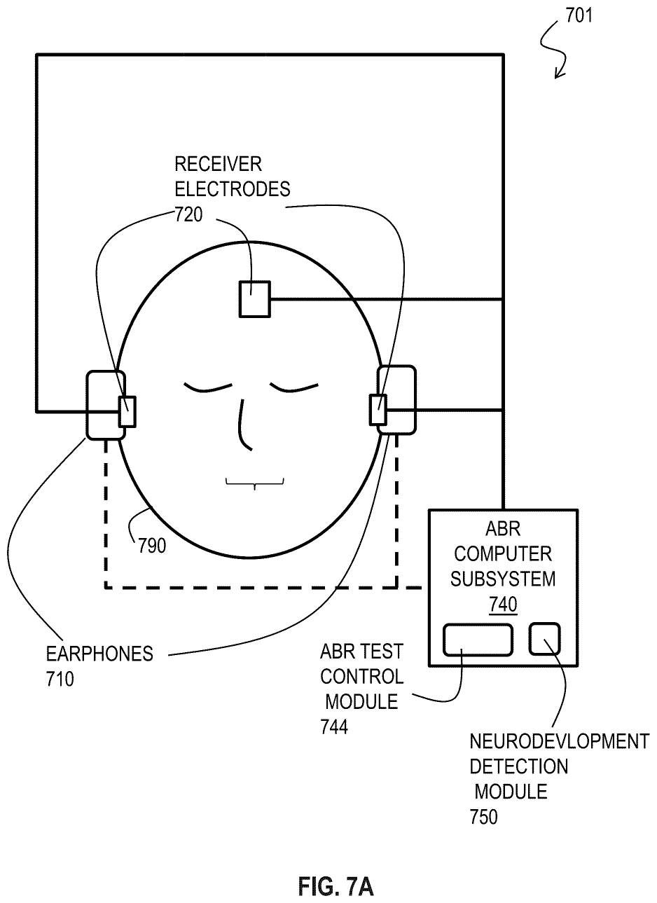

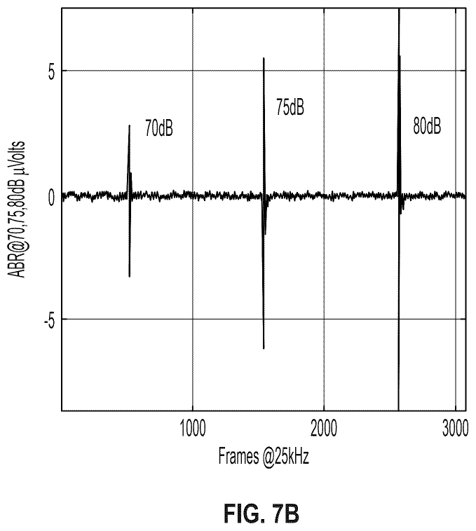

In some embodiments, the system 101 includes an Auditory Brainstem Response (ABR) test subsystem 120, as described in more detail below with reference to system 701 in FIG. 7A. In these embodiments, the neurodevelopment detection module 150 incorporates data from the ABR test subsystem 120. In various embodiments, such ABR test data includes the timing and volume of one or more auditory stimulation signals, the timing of any primary peaks, and one or more properties of micropeaks in the electrical signal recorded before and after such auditory stimulation signals, as described in more detail below. As used herein, a micropeak is an electrical signal peak between successive minima accomplished on the time scales of a tens of microseconds to hundreds of milliseconds.

In some embodiments, the neurodevelopment detection module 150 includes one or more data files or databases (not shown) that hold data that indicate one or more predetermined distributions of micromovement spikes or ABR electrical micropeaks, or both, in one or more populations of control individuals, each population associated with a different neurodevelopmental condition, such as a later diagnosis of ASD or no ASD.

Any known algorithm or software application can be used as the video pose module 144. Several different algorithms for pose estimation have been published over the past decade (e.g., OpenPose (Cao et al., 2017), DeepLabCut (Mathis et al., 2018), DeepPose (Toshev et al., 2014), DeeperCut (Insafutdinov et al., 2016), AlphaPose (Fang et al., 2018), ArtTrack (Insafutdinov et al., 2017)). Using these algorithms, it is possible to take advantage of pretrained networks that are freely available, or train new networks customized for various research or clinical needs. For example, a commonly used pretrained network is the human pretrained demo of OpenPose that includes key points of the body, feet, hands, and face and has been used in several recent studies for quantitative analysis of human movement, including VideoPose.

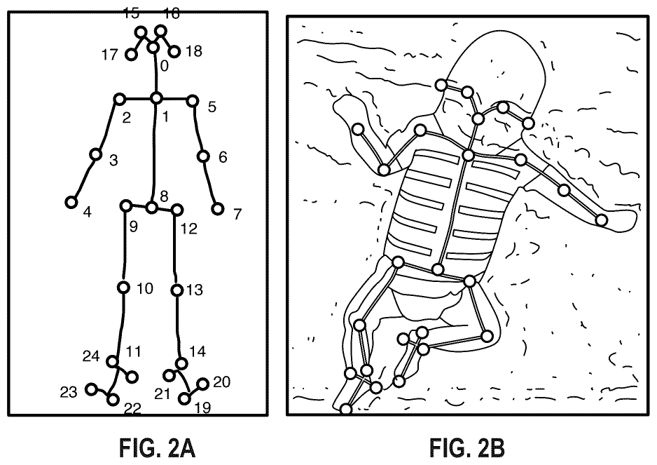

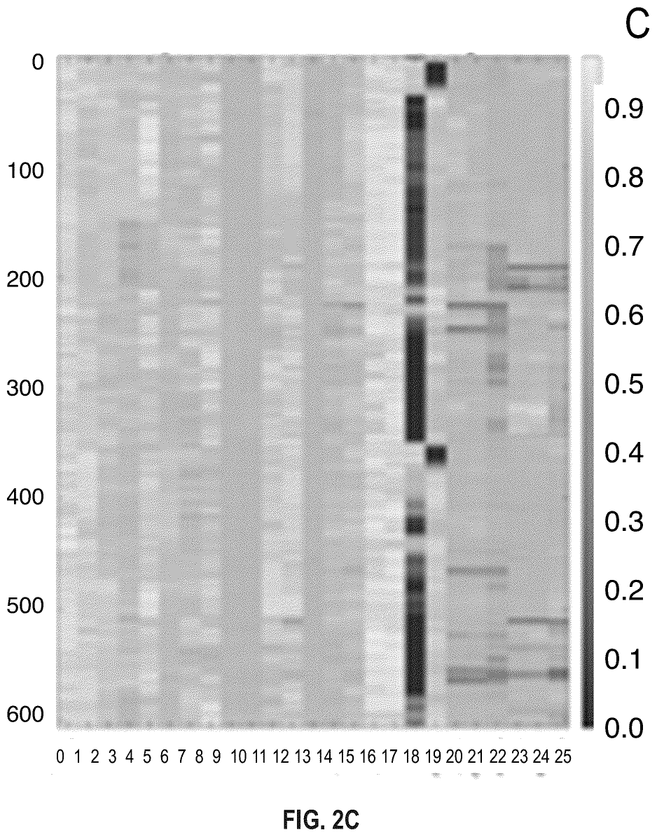

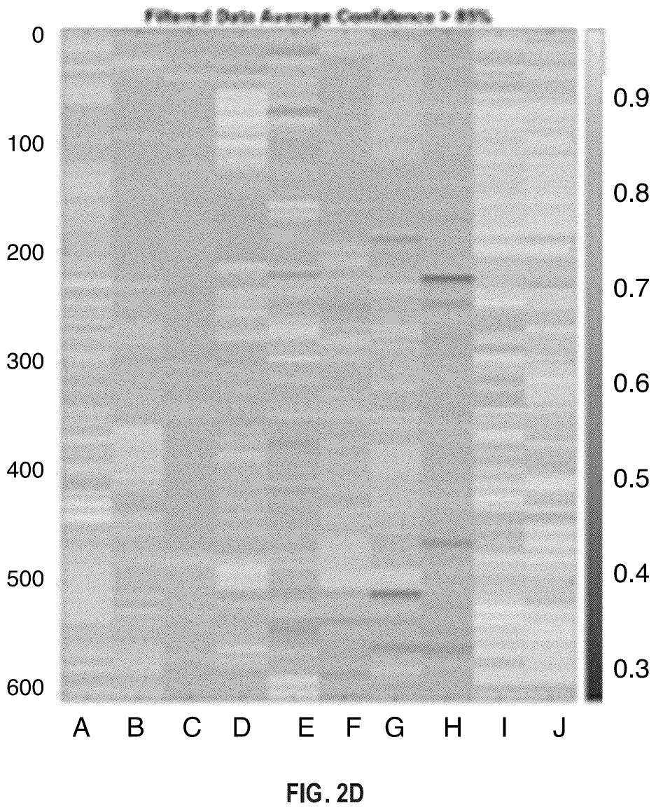

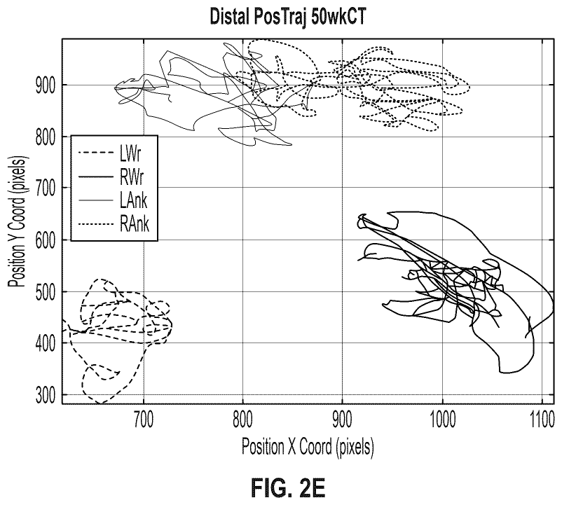

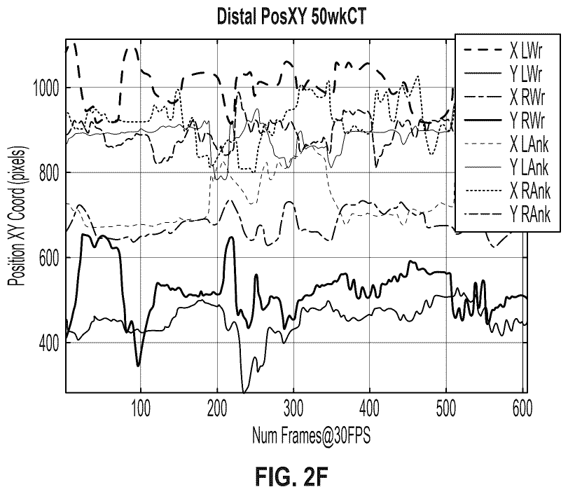

FIG. 2A is a block diagram that illustrates an example pose structure for a neonate, according to an embodiment. This pose estimation model was used by OpenPose to derive 25 anatomical positions or joints (collectively called “joints” hereinafter) corresponding to nose/head (0), chin/neck (1), right shoulder (2), right elbow (3), right wrist 4, left shoulder (5), left elbow (6), left wrist (7), pelvis/trunk (8), right hip (9), right knee (10), right ankle (11), left hip (12), left knee (13) left ankle (14), right eye 15, left eye (16) right ear (17), left ear (18), left foot 919), left toes 920), left heel (21), right foot (22) right toes (23) and right heel (24). These joints are grouped into distal joints (wrists “Wr” and ankles “Ank”, feet), proximal joints (head, neck, shoulder, trunk, hips) and articulated joints (elbows and knees) joints FIG. 2B is a diagram that illustrates an example superposition of the pose structure of FIG. 2A onto a video frame of a subject neonate, according to an embodiment. The OpenPose pose algorithm successfully associated each of the 25 joints with a pixel location on the two-dimensional video frame.

In some embodiments, the pose algorithm also provides a confidence metric that indicates the probability that the selected location is the specified model joint. FIG. 2C is a diagram that illustrates an example confidence matrix for the pose algorithm to detect each of 25 joints in each five-frame bin of a video clip including 600 frames (20 second clip at 29 frames per second, fps), according to an embodiment. The light shades are high confidence and the dark shaded indicate lower confidence as indicated by the vertical shade scale to the right of the matrix. The horizontal axis indicates the joint number and the vertical axis indicates the frame number from top (frame 0) to bottom (frame 600). Joints detected with high confidence are subjected to further analysis for micromovement spikes (MMS). For example, only joints with an average above 90% confidence are retained for MMS analysis. FIG. 2D is a diagram that illustrates an example confidence matrix for the pose algorithm to detect each of 10 joints detected with at least 90% confidence in each five-frame bin of a video clip including 600 frames (one minute clip at 10 frames per second), according to an embodiment The ten joints retained in this example include: A left wrist; B right wrist; C left ankle; D right ankle; E head; F neck; G left shin; H left eye; I right eye; J left knee.

The next step is to plot the two-dimensional trajectories of each retained joint. FIG. 2E is a graph that illustrates example two-dimensional trajectories (X-Y plane) of four distal joints (left and right wrist and ankle) from the pose algorithm, according to an embodiment. The horizontal axis indicates pixel number in the X direction, and the vertical axis indicates pixel number in the Y direction. The trajectories represent the various positions taken by each of the four distal joints for one neonate subject of a control (CT) population during a 20 second video clip recorded at 29 video frames per second. FIG. 2F is a graph that illustrates example 8 one-dimensional profiles (X or Y coordinates plotted separately) of four distal joints (left and right wrist and ankle) from the pose algorithm, according to an embodiment.

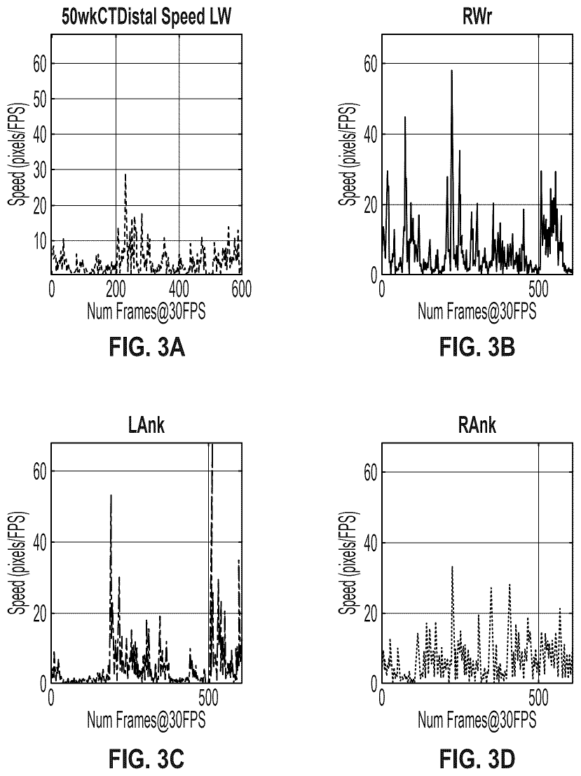

FIG. 3A through FIG. 3D are graphs that illustrate example speed micromovement profiles of four distal joints (left and right wrist and ankle, respectively), according to an embodiment. In each, the horizontal axis indicates number of frames at 29 fps, and the vertical axis indicates scalar speed in pixels per frame. Scalar speed is pixel distance, d_{P}, per time for one frame, where d_{P} is given by Equation 1.

$${d}_{P} ( i ) = \sqrt{{( {X}_{i} - {X}_{i - 1} )}^{2} + {( {Y}_{i} - {Y}_{i - 1} )}^{2}} \qquad ( 1 )$$

As can be seen in FIG. 3A through FIG. 3D, there are micromovement spikes (MMS) in scalar speed profiles for each of the distal joints. In other embodiments, other parameters of micro movement can be used, such as scalar acceleration, X-speed, Y-speed, X-acceleration, Y-acceleration, among others.

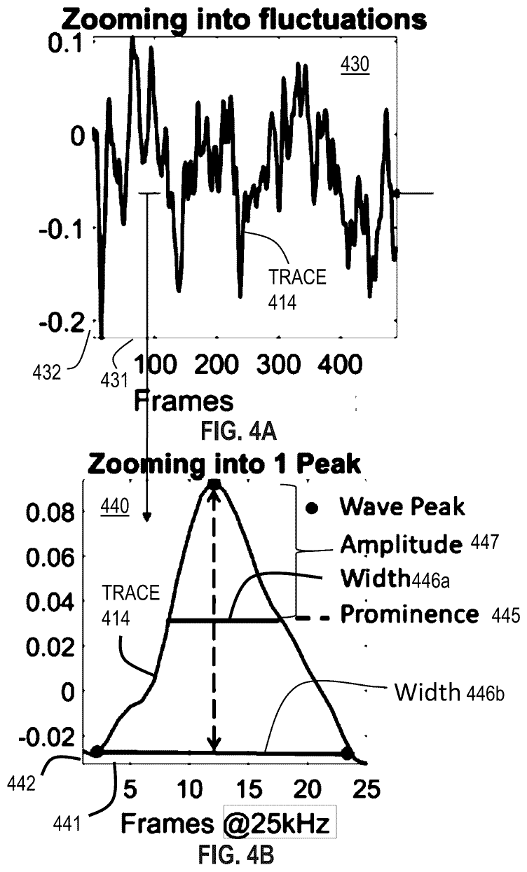

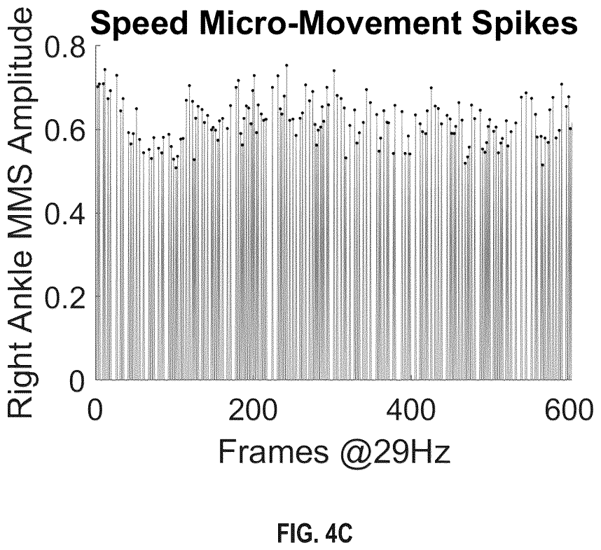

FIG. 4A and FIG. 4B are graphs that illustrate example properties of micromovement spikes, such as might be found in micromovement profiles such as depicted in FIG. 3A through FIG. 3D, above), according to an embodiment. For each graph, the horizontal axis indicates a number of frames, and the vertical axis indicates the amplitude of a movement property, such as scalar speed or scalar acceleration, among others. Trace 414 include multiple fluctuations. A micro movement spike (MMS) is considered to be the maximum movement value between successive minimum movement values. FIG. 4A shows multiple MMS along trace 414. FIG. 4B shows a single MMS from FIG. 4A. The single MMS in FIG. 4D at frame 12 along the horizontal axis has properties that include a prominence 445 indicated by a dashed line segment from the largest minimum value on each side of the peak to the maximum value, an amplitude of half the prominence value, a width 446a at half height, and a width 446b at the base which is at the value of the maximum of the two minima bracketing the MMS.

In some embodiments, the MMSs in the profile are normalized by the value of the largest prominence of all the MMS in the profile being processed. In some embodiments, the normalization of any MMS is computed using Equation 2.

$$P {n}^{*} = Pn / [ Pn + Avg ( P min 1 , P min 2 ) ] \qquad ( 2 )$$

Where Pn is the prominence of the nth MMS, Pn* is the normalized prominence of the nth MMS, and Avg(Pmin1, Pmin2) is the average of all the points between the two local minima Pmin1 and Pmin2, including the local maximum (MMS) Pn When the Avg(Pmin1, Pmin2) is small compared to the prominence of the nth MMS, the value of Pn* approaches 1. Such normalization tends to scale out allometric differences in length, weight, anatomical size, and other characteristics for individuals in the same group (e.g., same age or stage of development or neurodevelopmental condition or combination).

The normalized prominences of the MMS for one joint are plotted in FIG. 4C. FIG. 4C is a graph that illustrates example normalized micromovement spikes (MMS) in the micromovement profiles, according to an embodiment. The horizontal axis indicates frame, and the vertical axis indicates normalized prominences of MMSs. Each MMS is plotted at the frame where it occurs with a normalized prominence value between 0 and 1.

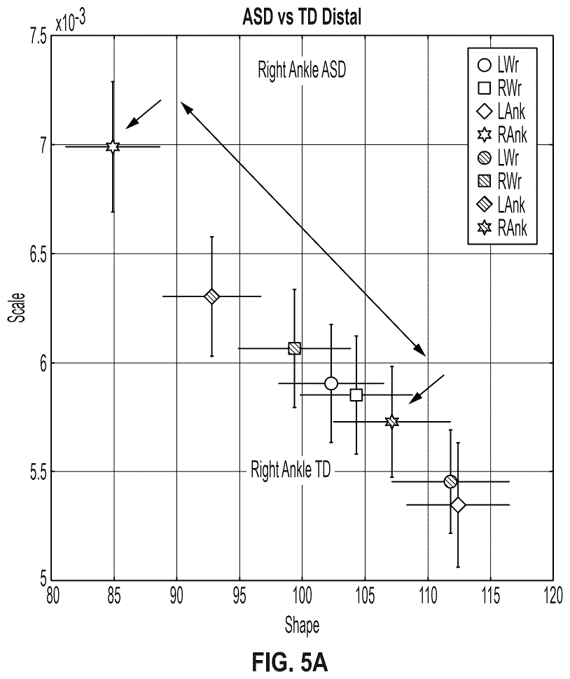

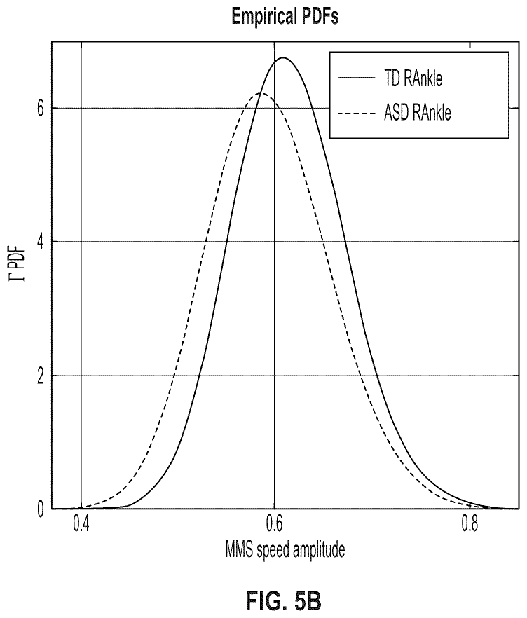

FIG. 5A is a graph that illustrates example Gamma Function parameters values for distributions of normalized MMS in the four distal joints, which detect differences in neurodevelopment, according to an embodiment. The horizontal axis indicates values for the Gamma shape parameter and the vertical axis indicates values for the Gamma scale parameter. Points are plotted for four distal joints in two populations. The circle symbol indicates the left wrist, the square the right wrist, the diamond indicates the left ankle and the star indicates the right ankle. The top four symbols represent the ASD population and are connected by callout lines to the plotted points. The other points represent the typically developing (TD) population for at least some joints there is a noticeable distinction between the parameter values (and thus the distributions) of the normalized MMS for the ASD population from the TD populations. The greatest difference between the two populations is in the Gamma parameter values of the right ankle emphasized by the arrows.

FIG. 5B is a graph that illustrates example distributions of normalized MMS in the right ankle based on the Gamma function parameter values plotted in FIG. 5A, which detect differences in neurodevelopment, according to an embodiment. The horizontal axis indicates the speed profile MMS amplitude values and the vertical axis indicates the relative occurrence values. The ASD population has a broader distribution with a lower mode occurrence at a lower MMS amplitude in the speed profile.

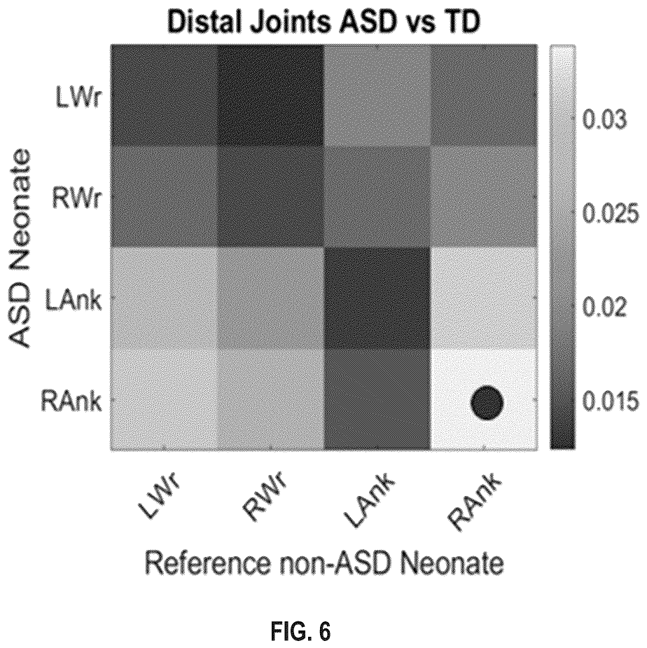

FIG. 6 is a block diagram that illustrates example distance between distributions of micromovements for the two populations at each pair of distal joints, according to an embodiment. The horizontal axis indicates the TD (e.g., non-ASD) neonate population for four distal joints, and the vertical axis indicates the ASD population for four distal joints. The shading scale indicated the difference between the distribution that characterize the MMS between each pair of joints. This distance can be calculated in any known manner. For purposes of FIG. 6, the Earthmovers distance is used. As can be seen, the biggest difference is between the distribution of MMS in the right Ankle of the non-ASD population and the distribution of MMS in the right Ankle of the ASD population.

In some embodiments, the MMS data is combined with micropeaks in ABR data to further distinguish different neurodevelopmental populations. FIG. 7A is a block diagram that illustrates an Auditory Brainstem Response (ABR) test setup, according to an embodiment. An Auditory Brainstem Response (ABR) test measures the reaction of the parts of a child's nervous system that affect hearing. The ABR test measures the hearing nerve's response to sounds and is a routine test for neonates, from premature babies to neonates up to six months in age to children up to seven years in age. The ABR test is typically performed when the child is sleeping or lying perfectly still, relaxed and with his or her eyes closed, and may involve anesthesia. Three to four electrodes are placed on a child's head and in front of his or her ears and connected to a computer. As sounds are introduced through earphones over the child's ears, the electrodes measure how the child's auditory nerves respond to them. Video captured before and during such ABR tests have been utilized to detect micromovements that correlate with or augment or otherwise detect other neurodevelopmental disorders.

FIG. 7A is a block diagram that depicts an Auditory Brainstem Response (ABR) test system 701, according to an embodiment. Although a head of a subject 790 is depicted, the subject 790 is not part of the system 701. The system includes earphones or other speakers 710 that are configured to be placed over the ears of a subject and deliver sound to the ears of the subject 790. The system includes an ABR computer subsystem 740 in communication with the earphones 710 by wired or wireless connections depicted as dashed lines. The system also includes one or more electrodes 720 configured to be placed at corresponding locations on the skin of the head of the subject 790 to pick up electrical signals and transmit them to the ABR computer subsystem 740 by wired or wireless connections depicted as solid lines. The ABR computer subsystem includes hardware and software configured to perform as an ABR test control module 744. The function of the control module 744 is to operate the earphones or speakers to deliver an auditory signal such as one or more tones of a predetermined frequency and volume. The function of the control module 744 is also to record on a computer-readable medium or display, or both, the electrical signal detected at each electrode 720.

According to various embodiments, the system 701 includes a neurodevelopment detection module 750 comprising hardware and or software and configured to detect attributes of the electrical signal from electrodes 720 that can be used to infer a condition of the subject 790 by comparing those electrical signals to electrical signals associated with one or more populations of individuals with known neurodevelopmental conditions. Module 750 is configured to implement one or more of the methods described herein. Each of modules 744 and 750 include one or more data structures (not shown explicitly) to hold data. For example, module 744 includes one or more data structures that hold data that indicates the auditory signals to be delivered to earphones 710. For example, module 750 includes one or more data structures that hold data that indicate electrical signals received from electrodes 720, or distribution of properties of electrical signals associated with one or more populations, or some combination.

Although processes, equipment, and data structures are depicted in FIG. 1 and FIG. 7A as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

FIG. 7B is a block diagram that illustrates an ABR test electrical signal recorded after each of three sounds of increasing volume, according to an embodiment. The horizontal axis indicates time in frames of 1/25000 of a second, so that one millisecond corresponds to 25 frames. There is an electrical measurement at each frame from at least one electrode. The vertical axis indicates the strength of the electrical signal voltage in micro Volts (μV, 1 μV=10⁻⁶ volts). A few milliseconds after each auditory stimulation, there is a large excursion of voltage corresponding to the arrival of one or more vertices. Each auditory stimulation has a different acoustic volume, indicated by decibel (dB) level. The first auditory signal is at 70 dB and elicits a first voltage response in the electrical signal near the 500^{th} frame. The second auditory signal is louder at 75 dB and elicits a larger second voltage response in the electrical signal near the 1500^{th} frame. The third auditory signal is loudest at 80 dB and elicits a largest third voltage response in the electrical signal near the 2500^{th} frame. The time of the auditory signal is not shown but is presumably within about 10 milliseconds (250 frames) of the electrical response.

During an ABR test, the auditory signals and electrical response shown in FIG. 7B are repeated multiple times, typically 10 or more. The large electrical excursions and the associated vertices are reviewed to determine that the subject has a normal or abnormal hearing response appropriate for the age of the fetus, neonate or child.

In various embodiments, microscale peaks on time scales from several microseconds to hundreds of milliseconds, in the electrical signal of FIG. 7B are processed, similarly to the MMS described above, to detect or otherwise quantify the neurodevelopment condition of the subject beside hearing acuity.

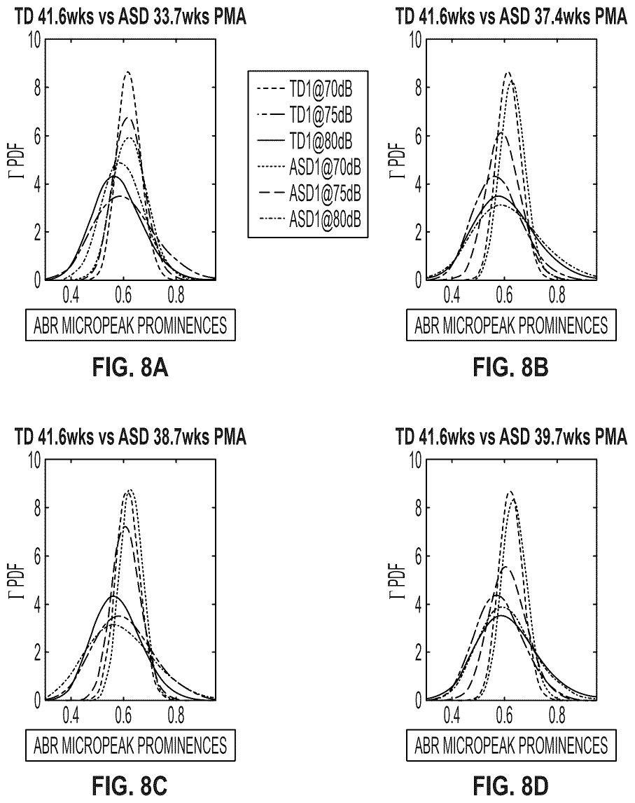

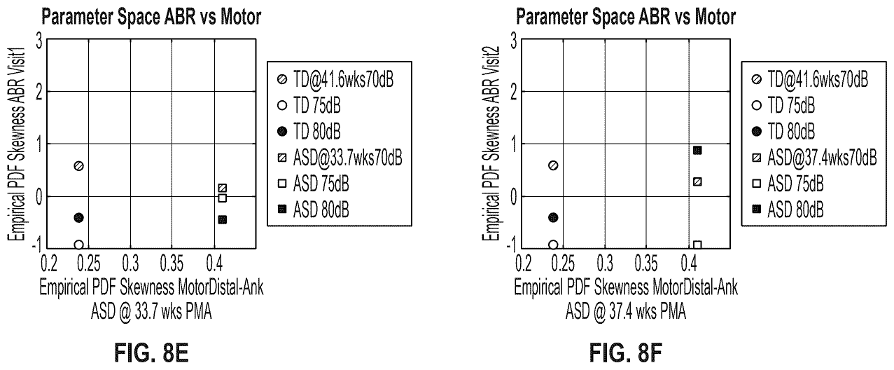

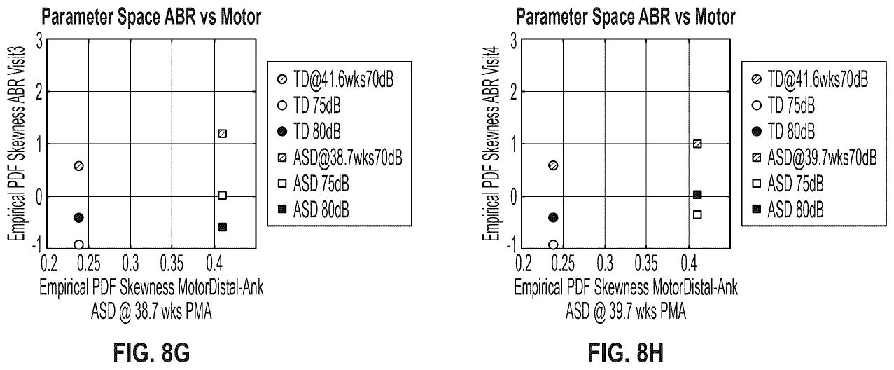

FIG. 8A through FIG. 8D are graphs that illustrate example differences in distributions of micropeaks in ABR electrical signals between ASD and nonASD populations for all three sound volumes, according to an embodiment. In each plot the horizontal axis indicates prominences of micropeaks in the electrical signal recorded during an ABR test and the vertical axis indicates the number of occurrences in relative units. In each plot three distributions are plotted with solid lines to indicate the distributions of an individual from the typically developing (TD) population at three different volume levels. Near each solid line distribution, another distributions is plotted with dashed lines to indicate the distributions of an individual from the ASD population at three different volume levels. In each of the four plots the TD distributions are the same representing the TD individual at 41.6 weeks Post-menstrual age PMA. The four plots in FIG. 8A through FIG. 8D differ in showing the distributions for the ASD individual at 33.7 weeks, 37.4 weeks, 38.7 weeks and 39.7 weeks, respectively.

These plots demonstrate that the methods described herein enable the detection of differences in PDFs, and also dynamically track these signatures, as they change over time and shift values on the Gamma parameter plane. This is shown in FIG. 8A through FIG. 8D for Gamma PDFs empirically estimated from the Auditory Brainstem Responses (ABR) of these babies using fluctuations in amplitude of the ABR as measured by the peaks' prominences. Longitudinal data for the one diagnosed with ASD with respect to the TD baby are shown for 3 levels of auditory signals (70, 75, 80 dB) for different visits to the clinic. For ASD baby the time at each visit is shown on and the TD had one visit at 41.6 weeks

FIG. 8E through FIG. 8H are graphs that illustrate example differences in skewness ABR micropeaks compared to skewness in MMS between ASD and nonASD populations for all three sound volumes at four different ages of neonates, according to an embodiment. The four ages of the ASD child are 33.7 weeks PMA, 37.4 weeks pma, 38.7 weeks PMA and 39.7 weeks PMA. In each graph the horizontal axis indicates skewness of the empirical probability density functions (PDFs) representing the distributions of MMS. The vertical axis indicates skewness of the empirical probability density functions (PDFs) representing the distributions of ABR electrical micropeaks. While there is overlap in the skewness of the ABR micropeak distributions, there is clear separation in the skewness of the MMS distributions at all ages of the ASD child.

More subtly, the ASD baby shows a trend of increasing skewness along both the ABR (central nervous system) and Motor (peripheral nervous system) responses. First visit differentiates the babies in the range of responses (narrower in the ASD one), then a systematic increase in skewness of the distributions of ABR is noted, with maximal separation at 39.7 weeks in ASD with respect to the TD visit. The motor data only has one data set at the last visit, but it too shows an increase in skewness (away from the symmetric Gaussian that all biorhythms tend to in neurotypicals.) Indeed, this increase in skewness is often accompanied by increase in the noise to signal ratio (NSR) given by the ratio of Gamma parameters b/a, where a is the scale parameter and b is the shape parameter, for ASD child. This is also shown in children older than 3-years.

In general, differences in MMS distribution properties are expected for other neurodevelopmental conditions or disorders, either directly or as a surrogate for ABR micropeaks distributions. Using empirically estimated statistics instead of a priori adopted probability distribution functions (PDFs), leads to detection of anomalies, often by 3 months of age. As babies grow, their rates of development of motor control towards volition, linearly correlates with their growth rates on a log-log scale, a relationship that breaks down when neurodevelopment goes awry. Similarly, by 4 years of age, an infant's volitional control of pointing motions matures and transitions from the exponential to skewed distributions with heavy tail, only to approach a Gaussian range during college age. This maturation follows a power law across human aging. In autism however, maturation of noise-to-signal ratio (NSR) from voluntary motions is stunted. Individuals across 3-30 years old remain closer to the memoryless, random exponential regime, with high NSR in the moment-by-moment fluctuations of the speed that the hand traces during naturalistic motions. Importantly, across human biorhythms, there is a tight power law relation fitting the scatter of empirically estimated Gamma parameters (expressed on the [shape, scale]-Gamma plane). This tight fit reduces the parameter of interest to one: the scale, denoting the NSR (Torres et al., 2013d). This not only offer a target for treatment that is traceable over time, but also able to dampen high levels of random noise in the biorhythms of the nervous systems, evoking spontaneous autonomy and the sense of action ownership, by identifying the best sensory-motor capabilities and predispositions of the child, in a personalized manner.

In neonates, movement variability as captured by distributions of MMSs, offers hope for much earlier detection of problems involving the building blocks of social interactions. Abstract social cognition in human infants matures later than well-organized, well-coordinated motor patterns. As such, assessing neonatal movement patterns in the NICU, along with the quality of their variability, can offer an earlier window into brain development, particularly when paired with Auditory Brain Response (ABR) data routinely collected at the NICU (Karmel and Gardner, 2005).

Indeed, the transition of motor patterns from writhing movements, to fidgeting movements, to well-organized, more predictive movements, precedes intentional motions appearing after three months of age. This progression constitutes a hallmark of human neurotypical development absent from other primate species. From the 28th week of gestation to the third month of extrauterine life, something special about the verticalization of the head-trunk later facilitates upright walking against gravity. This evolution of human motor control marks precise maturational phylogenetically ordered milestones that now can be digitized and characterized with high precision. Understanding this early evolution of brain-body development identifies anomalies in the building blocks of social cognition under a new unifying framework connecting the central and the peripheral nervous systems through stochastic analyses of standardized time series data.

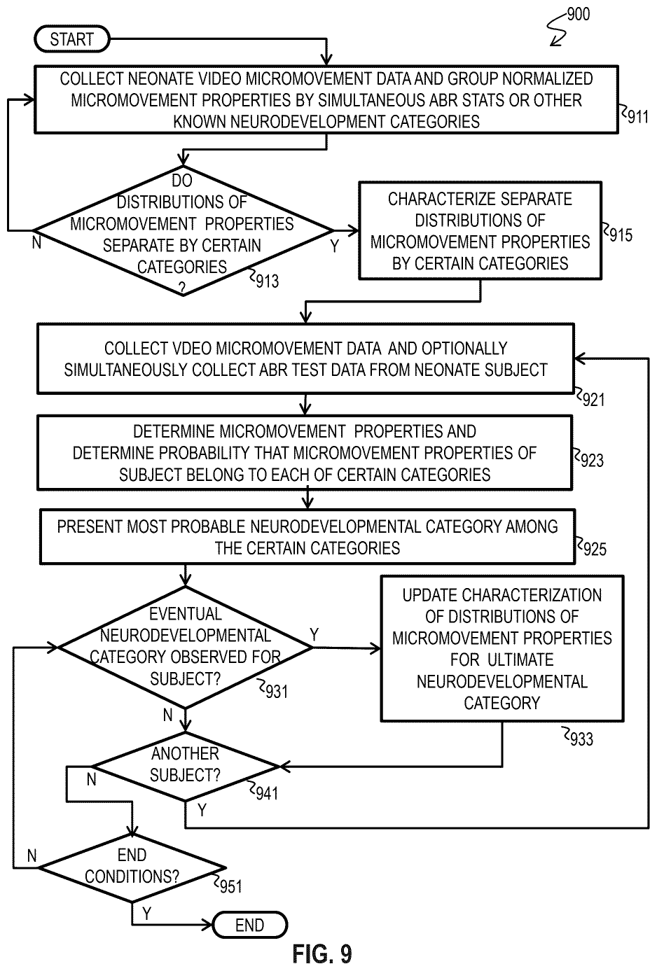

FIG. 9 is a flow diagram that illustrate an example method 900 to detect differences in neurodevelopment of a subject based on distributions of MMS, according to an embodiment. Although steps are depicted in FIG. 9 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways

In step 911, neonate video data, with or without ABR test data, is collected for a plurality of individuals at various ages, from fetal ages to premature ages, to neonates, and on into childhood. The video data is transformed to MMS data for one or more joints or anatomical landmarks (collectively called joints herein) using any known pose algorithms, such as OpenPose. Each individual is further tracked and eventually assigned to one of several neurodevelopmental categories, each reflective of a certain neurodevelopmental condition, such as typical or ASD, among others. For each neurodevelopmental category, the distribution of one or more properties of MMSs with or without distributions of one or more properties of ABR micropeaks, is determined. Any property can be used for the distribution of either MMS or ABR micropeaks, including the prominence, the amplitude, the width at half height, the width at base, or the inter-peak latency, or some combination. For example, an empirical histogram of one or more properties of MMSs or micropeaks is determined, e.g., for each of one or more peak properties, and the mean, variance and skewness and kurtosis or some combination of the empirical distributions are determined, or a functional fit, such as a Gamma function fit, to the empirical histogram is performed, and the values of the functional fitting parameters are stored for each property and neurodevelopmental category.

In step 913, it is determined whether there are two or more categories with statistically significant differences in distributions. Any method may be used to determine the statistical significance of differences in two distributions. Example methods include the Kolmogorov-Smirnov test, the Jensen-Shannon divergence, which is an extension of the Kullback-Leibler divergence (one sided), the Wasserstein distance (also known as the Earthmover's distance, EMD) and an equivalent UNIFRAC distance used in the microbiome world, and classical methods for non-normal cases such as the Kruskal-Wallis test (non-parametric ANOVA1) or Friedman test (ANOVA2), and the two-sided Wilcoxon rank sum test, among others. If not, control flows back to step 911 to continue to collect ABR test data for different categories of neurodevelopment. When a significant difference is found among two or more distributions of certain categories then control passes to step 915.

In step 915, the certain categories of neurodevelopmental conditions are stored along with data indicating their distributions, such as an empirical histogram, or mean and variance and skewness of an empirical histogram, or the values of one or more functional fitting parameters, such as values for shape and scale of the Gamma Function, or some combination. Any method may be used to store this information, in one or more data structures, such as a flat file or files or a relational database. These are the predetermined distributions of certain neurodevelopmental categories, such as the distribution depicted in FIG. 5B for typical (TD) and ASD categories, or the plots of shape and scale parameter values for the Gamma Function, such as shown in FIG. 5A.

In step 921, video data is collected from a subject, such as during a routine clinical visit for testing hearing acuity of a neonate. In some embodiments, the collection of step 921 is initiated for other reasons, e.g., explicitly to determine the neurodevelopmental conditions of the subject. Step 921 includes a sufficient amount of video data (with or without ABR test data) to obtain a good distribution of the properties of MMSs (with or without ABR micropeaks).

In step 923, the distributions of one or more MMS properties, are compared to the predetermined distributions of MMS properties stored for either the certain neurodevelopmental categories or for the distribution of ABR micropeaks that serve as a surrogate for the neurodevelopmental category. As a result, the probability that the distribution is associated with each ABR micropeak distribution or neurodevelopmental category is determined. Any method may be used to determine this. For example, the z-test or t-test or Anderson-Darling statistic or a similarity statistic, or Earth-movers distance can be used, among others, as described above.

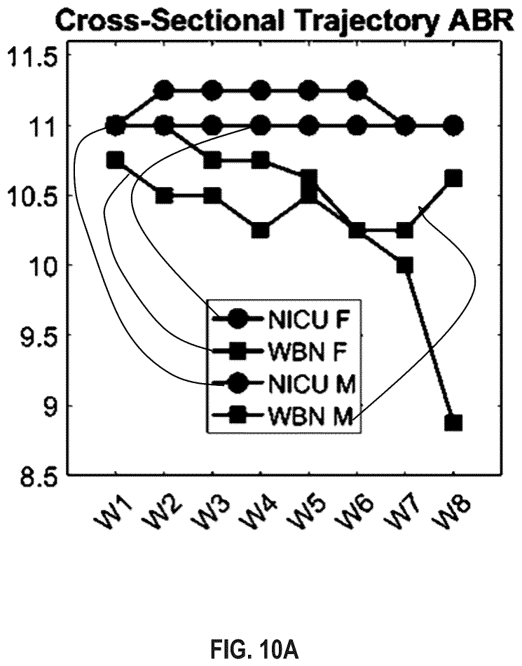

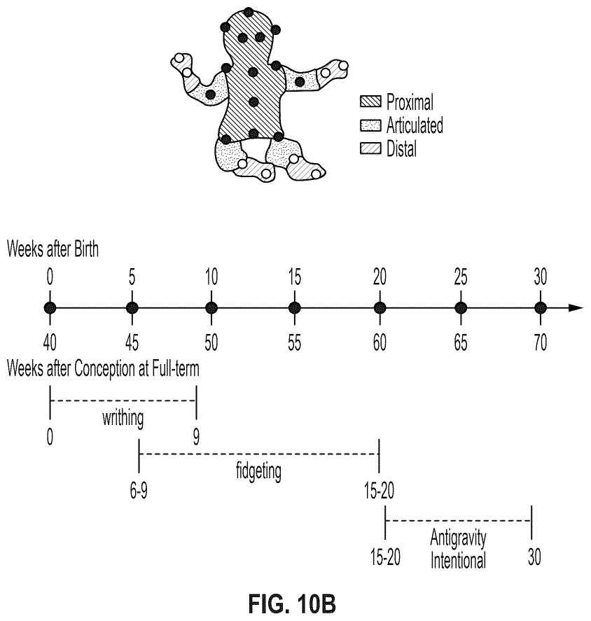

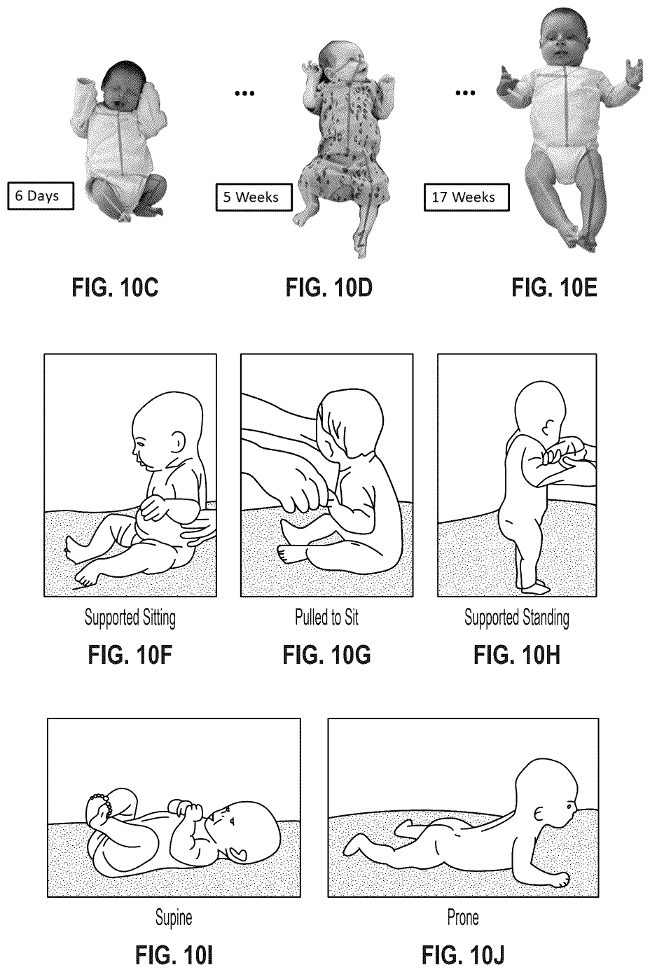

In step 925, the most probable neurodevelopmental category is presented, e.g., to the doctor, clinician, or other caregiver. For example, the category is presented on a computer display. In some embodiments, the presentation includes the probability of the category. In some embodiments, several categories and associated probabilities are displayed. This serves to notify the doctor, clinician or other caregiver of the usefulness of further testing, observation, or treatment for the subject. In some embodiments an age chart is presented that gives the one or more features of the PDF for two or more categories at different ages, similar to the chart depicted in FIG. 10A

FIG. 10A is a graph that illustrates an example growth chart presentation for showing changes in a property of a distribution with age for two different populations, according to an embodiment. This embodiment compares neonatal intensive care unit (NICU) vs Well Baby Nursery (WBN), using clear peaks evident in ABR test data. In general, WBN neonates are trending down in latency, but NICU are steady, not changing much for either females or males. Different temporal trajectories are obtained for different parameters, but FIG. 10A is based on the minimum latency of the ABR electrical signal. One can tell which babies are stunted because the latencies are not decreasing. Using such a growth chart, one gets different trajectories for different MMS with or without micropeak PDF parameters; and any such trends for any population differences can be presented in a manner similar to FIG. 10A.

In some embodiments, the stored data is updated as the subject's eventual development is tracked. In step 931, it is determined whether the eventual neurodevelopmental category is observed for the subject. For example, it is determined that the subject undergoes typical neurodevelopment. If not, control passes to step 941 to determine if there is another subject. However, if the eventual category is observed for the subject, then control first passes to step 933 and then to step 941. In step 933, the characterization of the distributions of MMS properties 9 with or without distributions of ABR micropeaks properties) for the neurodevelopmental category of the subject previously stored during step 915 is updated during step 933. The observed distribution of each MMS property for the current subject at the various ages or stages of development are added to the stored measurements at those ages or stages. Thus, the system eventually learns a more comprehensive characterization of the neurodevelopmental category.

In step 941, it is determined whether there is another subject for video recording. If so, control passes back to step 921 and following, described above. If not, control passes to step 951.

In step 951, it is determined if end conditions are satisfied, e.g., that the system is to be powered down or to halt tracking the development of any subjects. If not, control passes back to step 931, described above. If so, then the process ends.

### 2. Example Embodiments

In an example embodiment, access was obtained to a vast data repository dating back to 1994 until present time. This repository includes longitudinal and cross-sectional information collected in the NICU from preterm babies, some eventually diagnosed with autism (Karmel et al., 2010). Learning (prospectively) about their longitudinal motor transitions and maturation, in combination with the early clinical data at birth, such as Apgar scores, cranial ultrasound, and the ABR data, enable the building of likelihood ratios, forecasting cognitive and social differences in autism much earlier than is currently done. These biorhythmic motor- and brain-based clinical criteria (e.g., MMS and ABR micropeaks) then provide a new objective, very early detection method. This new method leverages motor dynamics to track neurodevelopmental changes accompanying physical growth and brain maturation. More precisely, the central nervous system (CNS) signals represented by the ABR signals help monitor brain function and development, while the peripheral nervous system (PNS) represented by the micromovement motor data helps monitor kinesthetic reafferent feedback. Tracking these patterns in healthy controls vs. babies who eventually developed autism help make visible those ambiguous patterns of neuromotor control development and match them to atypical ABR also associated with such categories of neurodevelopment. Upon characterizing their CNS-PNS relationship, one can use the very early (developing) motor signal as a proxy detector of the very early ABR signal of brain development and eventual neurodevelopmental diagnosis.

Understanding movement and its sensation in autism can more generally build support for the autistic system, by relieving the brain from the excessive cognitive load that having to explicitly think about motor coordination and motor control imposes, when faced with new situations. We have found and confirmed from autistics themselves that there is a disconnect between mental intent and physical action in autism. The physical realization of the intended act is difficult at many levels. From an early age, the person spends most mental energy coping with excess (undesirable) involuntary motions invisible to the naked eye of the observer. Excess noise and randomness in the motor reafferent feedback to the brain, necessarily forces the autistic brain to closely attend to every aspect of the action that it intends to perform. This, in turn, overloads the brain with cognitive effort to attain simple goals in daily life. We know this because we have quantified its neural correlates in the brain activity from deafferented patients whose signatures of motor noise resemble those of many autistics. Part of the problem is the typical reactivity of the nervous system to prompting. We discovered that in autism, externally bringing intent into full awareness (through prompting) in the presence of noise, amplifies uncertainty in the feedback to the brain. Relieving the brain from such cognitive loads involves the use of movements that occur largely beneath awareness.

In this sense, endogenously self-generated movements that in neonates are precursors of intended acts, could facilitate the study of the process of acquiring embodied cognition from an early age, to strengthen the building blocks of social interactions and communication disrupted in autism. This hypothesis has received support in the context of an experimental intervention that utilizes the motor variability from spontaneous (unintended) movements transpiring largely beneath awareness, to evoke self-exploration and self-discovery of perceptual and cognitive goals, without facing the type of reactivity that instructing to move evokes in autistics.

Our previous approach has evoked autonomous (self-discovered) cognitive control in 25 non-speaking autistics and matched neurotypical peers, by optimizing the systems capabilities, while minimizing anxiety.

It is known that after 3 months of age, the neurotypical infant initiates intentional movements. Tracking infants' neuromotor development prior to and after this period of life will help us characterize the transition from spontaneous movements to deliberate voluntary control that ultimately becomes automated and volitional. As explained above, underpinning the method of 900 of FIG. 9 is a realization that enabling and increasing automated self-generated motions can release the brain resources to explore and learn new goal-oriented tasks. This would open more room for social and cognitive development. By characterizing very early in infancy the developing relationship between central brain function and the quality of the peripheral motor feedback in neurotypical babies, it is expected that standard charts to measure deviation from these expected normative signatures can be provided.

In a longitudinal study, the NY State Office for People With Developmental Disabilities (NYOPWDD) group evaluated the contribution of initially abnormal neonatal ABR responses and 4-month arousal-modulated attention visual preferences, in ASD-diagnosed NICU graduates (Cohen et al., 2013). They compared NICU graduates with normal neonatal ABR to those with abnormal timing. The study found that those infants with abnormal ABRs and increased preference for higher rates of stimulation, were linked to an ASD diagnosis, and to rapid increases in ASD severity at 3 years of age. Furthermore, abnormal ABRs were associated with later reports of repetitive and ritualistic movements irrespective of 4-month preference for stimulation. These are now recognized as indexes of brainstem dysfunction implicated in autism (Cohen et al., 2013), a result that offers potential to subtype brainstem-related motor disruption, and infer brain damage, e.g. linked to cerebellar issues in autism (Wegiel et al., 2013). Indeed, prevalence of ASD has been reported in preterms 5-8% vs. 1-2% in the general population (Schieve et al., 2014).

Using their videos to extract movement variability patterns (FIG. 2A through FIG. 2F) and ABR activity (FIG. 8A through FIG. 8H), it is shown that prospective examination of movement in those babies subsequently diagnosed with ASD, in the presence of these earlier neonatal features, support the design of an early motor-based biomarker of neurodevelopmental derailment, potentially leading to an autism diagnosis. Furthermore, analyzing the ABR data under the same statistical platform as the motor data enables building a relationship to infer one from the other.

Continuous analogue data is converted to MMS or ABR micropeak streams by introducing the method depicted in FIG. 4A through FIG. 4C that extracts spikes from the moment-to-moment biorhythmic variations inherently present in natural behaviors. Such variations are otherwise discarded as gross data or noise under traditional analytical methods. Traditional methods assume a priori a theoretical distribution (e.g., the Gaussian distribution) and using a theoretical mean and variance, take grand averages that smooth out such fluctuations away from the assumed theoretical mean. The inventor's own earlier work showed that across biorhythms readout from the human nervous systems (across different ages) the distributions of parameters linked to the peaks of such data (including those from resting state) were skewed and non-stationary within neurodevelopmental time scales. The fluctuations could be converted to spike trains using methods that scale out neuroanatomical disparities across different ages, thus standardizing the multilayered biophysical data at the front end of any algorithm. These micro-fluctuations present across different biorhythms were coined micro-movement spikes (MMS).

Among signals used to characterize MMS in neonates, the physio (temperature), kicking and flailing-arm (kinematic) patterns were very informative of the longitudinal neurodevelopmental progression. They were registered using small triaxial accelerometers and gyroscopes that babies worn in leg- and arm-warmers.

These MMS signals paired with stochastic analytical methods to empirically estimate their signatures, helped us detect neurodevelopmental derailment by three months of age (Torres et al., 2016a). Specifically, maximum likelihood estimation (MLE) were used to approximate the shape and scale (dispersion) of the distribution family that best fits the MMS derived from fluctuations in spike amplitudes and inter-spike-interval timings of the original sensors' signals.

Separating physiologically relevant signal from instrumentation noise (Torres, 2017) enabled us to track important neural correlates in the behavioral domain and introduce to the cognitive and behavioral fields several techniques from the field of computational neuroscience, used to analyze and model cortical spiking data. Additional methods combine the kinematics from active behaviors with temperature data amenable to design various parameter spaces and derive physiological indexes predictive of perinatal and neonatal states. In the former, the daily data readings from wearables saved a comma patient who was pregnant, as we were able to anticipate the week of the baby's birthdate.

In applying the method 900 to these data, distributions are not theoretically assumed, but rather empirically estimated, and accompanied by stochastic analyses and non-linear complex dynamical systems approach, it is shown that one can detect very early anomalies. Indeed, when neurotypical development (a member of the TD population) is in place, as babies grow, their rates of development of motor control towards volition, linearly correlates with their growth rates on a log-log scale. Furthermore, this relationship breaks down when neurodevelopment goes awry, such as in members of the ASD populations. Likewise, when ABR patterns deviate from expected neurotypical ranges, the infant eventually (often) receives a diagnosis of ASD. Similarly, by 4 years of age, a child's volitional control of pointing motions matures and transitions from the exponential to skewed distributions with heavy tail, only to approach a Gaussian range during college. Importantly, the Gamma distribution fits the human data across ages to two parameters a and b (shape and scale, respectively) and has well defined moments, the mean μ and the variance σ given by Equations 3a and 3b, respectively.

$$μ = a b \qquad ( 3 a )$$

$$σ = a {b}^{2} \qquad ( 3 b )$$

Then, the noise to signal ration, NSR is given by Equation 3c.

$$NSR = σ / μ = b \qquad ( 3 c )$$

Tracking the NSR along with the skewness of the distribution, as babies develop, e.g., FIG. 8E through FIG. 8H, can uncover important longitudinal patterns to detect very early departures from normative patterns. Based on analyses of an example embodiment, atypical neurodevelopment in NICU graduates later diagnosed with ASD have different patterns of NSR across biorhythmic (time series) signals, compared to that of neurotypicals and other neurodevelopmental diagnoses.

Evidence along these lines comes precisely from ABR data, showing prolonged ABR wave latencies, a result that offers prospective subtyping brainstem-related disruption, also linked to cerebellar issues in autism). Further preliminary evidence (e.g. FIG. 8E through FIG. 8H) confirms that ABR and motor data are very valuable to detect very early anomalies and build maps conducive of standardized, dynamic look-up charts, such as depicted in FIG. 10A.

Data sets are post conceptional age (pca, measured in weeks at visit): pca00 is in hospital, pca01 is targeted for 44 weeks, pca03 at 52 wks, and pca04 at 4 months. Videoing was done at all visits listed until 4 months (sporadically thereafter)

TABLE 1
Data sets available for establishing predetermined
distributions for early detection of neurodevelopment
based on micromovements and ABR test data.

| | | Mean | Std. | Min | Max |
|---|---|---|---|---|---|
| | Num. | Age | Dev. | Age | Age |
| Variable | Babies | (weeks) | (weeks) | (weeks) | (weeks) |
| pca00 | 4,418 | 38.04944 | 2.593675 | 31.85714 | 47.42857 |
| pca01 | 3,249 | 44.60361 | 1.860768 | 40.28572 | 54.71429 |
| pca03 | 428 | 51.34146 | 1.662739 | 30.14286 | 56.85714 |
| pca04 | 2,827 | 58.79008 | 1.467772 | 53.71429 | 65.85715 |

According to an embodiment, these data are used in various steps of the method 900 of FIG. 9 to development early diagnosis of ASD using video data collected on neonates.

Step 911 includes curating thousands of longitudinal records collected from single babies, twins, triplets, siblings with autism diagnosis; organize the broader cross-sectional data in similar fashion and compile the (at least) 48 cases that were tracked beyond receiving an official autism diagnosis. Step 911 also includes deriving kinematics data from the video data, using pose estimation models employing deep learning techniques. In some embodiments, physiological transitions between spontaneous writhing, fidgeting and intentional movements are detected using the pose models and are stored in association with clinical scores and with ABR data.

In some embodiments, steps 913 and 915 include uncovering a standardized stochastic map (invariant to anatomical differences) of central nervous system (CNS) to peripheral nervous system (PNS) autonomy and PNS to CNS kinesthetic feedback quality, expressing dynamic transitions with maturation of neuromotor volitional control and physical growth. This map is expected to be analogous to the CDC/WHO growth chart but will contain dynamic neuromotor developmental milestones detecting the acquisition of volitional control of the central nervous system (ABR based signal) over the peripheral nervous system (bodily kinematics-based signal including MMS).

In some embodiments, step 925 includes creating a dynamic chart of developmental trajectories across thousands of babies and provide both longitudinal and cross-sectional characterizations of the neurodevelopmental transitions toward volitional motor control accompanying physical growth. Further, because in a subset of the data (over 48 records) the diagnosis of autism was obtained using the current gold standard, one can backtrack the earliest inflexion point of these stochastic trajectories and for the first time, determine objective, physiologically based early PNS-motor flags congruent with early CNS ABR flags that are congruent with the eventual clinical criteria.

The general approach behind this work posits that sensory-motor integration and sensory-motor transformations critical for socio-motor communication are impeded in individuals who go on to receive an autism diagnosis. The impairments are traceable in the NSR derived from the moment-to-moment fluctuations in the frequency and spatio-temporal domain parameters extracted from the person's biorhythms (such as MMS).

In some embodiments, the differences in distribution of MMS and ABR micropeaks are separable into classes of deliberate (voluntary) and spontaneous (incidental) movements that can be blindly detected in the data using our analytics. As mentioned, in autism, spontaneous motions can be used to help evoke voluntary control (at will), free of verbal prompting. As such, these movements, originating in early neonatal development, will help uncover a new (dynamic) detection and treatment avenue, with minimal reactivity from the nervous systems. This is so because these spontaneous movements occur largely beneath awareness.

In some embodiments, the stunted neurodevelopment observed in ASD at older ages is traced back to very early atypical neurodevelopment of CNS-PNS such as ABR and Body Kinematics MMS. Top-Down methods to test this hypothesis include age-dependent organization of the cohort, to identify those with a diagnosis of ASD and trace retrospectively the trajectory of their ABR and MMS motor patterns searching for identification of inflexion points transitioning from one stochastic regime to another, as expressed on various distribution parameter spaces of interest. These include parameter spaces of continuous families of probability distribution functions (PDFs) empirically estimated from the moment-by-moment micro-fluctuations in both the ABR and the video-motion derived MMS data. The Bottom-Up approach for such embodiments includes identification of automatically emerging clusters on parameter spaces blind to the clinical labels, followed by examination of these signatures, while considering the clinical labels. In cases where autism was diagnosed, the clinically identified individuals are labeled and using appropriate similarity metrics, its nearest neighbors are identified, all to be backtracked along the longitudinal trajectories of the parameters of interest.

In some embodiments, atypical neurodevelopment in NICU infants later diagnosed with ASD has different degrees/patterns compared to that of neurotypical and other neurodevelopmental diagnoses/delays. Here a top-down approach labeled the age-dependent ABR data according to category, such as the extent of the Germinal Matrix (intraventricular) injury hemorrhage detected by the Cranial Ultrasound and the Apgar scores. The MMS profiles are labeled accordingly. To identify clusters, multiple parameter spaces are derived, e.g., as depicted in FIG. 5A, and optimization methods are used to detect those parameters with maximal separation. Then the (bottom-up) blindly identified clusters are examined according to the top-down category labels. Babies who went on to receive the autism diagnosis are identified on these parameter spaces, to back track their stochastic trajectories and build a dynamic detection system from key transition points on those trajectories. Summary invariants (law-like relations) will be detected to further stratify the data accordingly.

In some embodiments, personalized biometrics integrating ABR and motion-kinematics (MMS) extracted from pose-estimation in videos, enable scaling their use to clinical and home settings. While ABR is commonly used at hospitals, video-based motion analyses to detect (endogenous) spontaneous transitions in motor milestones and subtype severity, further empower new parents with new tools at their disposal. In such embodiments, steps 921 includes using common smart phones to collect the video data and current cell-phone apps can send the data to clinicians for analyses. Furthermore, in step 923 algorithms described herein facilitate tabulations of milestones and subtyping to localize the baby's dynamic motor trajectories revealing the emergence of volitional control (or stunted neurodevelopment.) Combining these motor MMS data with physical growth measures will provide important dynamic biomarkers differentiating babies, while stablishing general universal commonalities of this very early development of the building blocks to scaffold social, communication and cognitive abilities.

The dynamic nature of the motor-based MMS biometrics and the time scales of the parameters of interest, enable the detection of change and its rate in response to neurodevelopment and to interventions. The characterization of the ABR CNS-signal via motor reafferent signal (PNS) using normalized MMS enable one to derive proxy biometrics of brain neurodevelopment and eventually bring them to NICUs where ABR may not be available.

The steps of method 900 can serve the dual purpose of detecting neurodevelopmental derailment and tracking neurodevelopmental change.

Furthermore, these steps contribute to close the current gap between cognitive and motor behavior. By identifying parameters that help autonomous regulation of motor control, it will be possible to free the baby's brain from the cognitive load of having to attend to aspects of motor behavior that are otherwise automatically controlled, thus giving way to building more abstract thought processes and creating room to properly develop an embodied theory of mind about others in a social context.

In some embodiments, video data is taken and processed independently of concurrent ABR data in clinical and non-clinical settings. Specifically in non-clinical settings a parent or other caregiver can use the method 900 with video data collected in a minute or so using any video capture device, including a smart phone such a depicted in FIG. 13, below. The parent would be instructed to pose the child in one or more ways appropriate to the child's age or development and collect video data for on the order of one minute (e.g., 0.1 minutes to 10 minutes) during step 921. Then the statistics of MMS are determined automatically by the video pose module 144 and neurodevelopment detection module 150 operating on the device, such as the smartphone, to perform step 923. The result is then presented to the parent, and optionally sent electronically to the neonate's physician or other clinician, during step 925.

In such embodiments, video data would be collected at various ages as depicted in FIG. 10B. FIG. 10B is a block diagram that illustrates various temporal epochs in a neonate's development suitable for at home video capture using the method of FIG. 9, according to an embodiment. Weeks after birth are indicated in a range from 0 to 30, corresponding to weeks after conception at full term from 40 to 70. In weeks after birth from 0 through 9, voluntary and involuntary writhing can be video recorded. In weeks after birth beginning about 6 to 9 and ending about 15 to 20 primarily voluntary fidgeting can be video recorded. In weeks after birth beginning about 15 to 20 and ending about 30 primarily voluntary intentional movement against gravity (labeled “antigravity”) can be video recorded. The avatar indicates proximal joints (head, neck, torso), distal joints (hand, wrist, foot, ankle), and articulated joints (elbow, knee) to be captured in the video taken. Each video is from about 0.1 to about 10.0 minutes in duration, with at least one minute duration being advantageous for good statistics

FIG. 10C through FIG. 10E are diagrams that illustrate example results of the pose module fit to a neonate in the supine position at 6 days, 5 weeks and 17 weeks, respectively, according to an embodiment. The pose module is able to track the movements of the same joints even as the scale of the neonate changes with age. The MMS normalization procedure also helps to correct for physical difference between different neonates at the same age or different ages of the same neonate.

FIG. 10F through FIG. 10J are diagrams that illustrate example postures for the parent or other caregiver to set the neonate for video-capture, according to an embodiment. FIG. 10F through FIG. 10H, show three postures suitable for increasing age or development of a neonate and include supported sitting, pulled to sit, and supported standing, respectively. Pulled to sit involves the infant laying on their back and being gently supported and pulled by the arms, to bring them to a sitting position. Supported sitting involves supporting the infant to remain in a sitting posture by providing support to the back. Standing/supported standing involves holding and/or supporting the infant to be in a standing position, until they are capable of doing so themselves. FIG. 10I and FIG. 10J show the supine (neonate lying on its back) and prone (neonate lying on its stomach) postures, respectively. The five postures of FIG. 10F through FIG. 10J have been classified as positions which convey information toward the potential for skill acquisition and motor development during the first year of life. The postures have been studied monthly in infants, and the transition in skill an infant has when performing such postures is informative of the development of the kinesiology underlying varying motor skills and/or motor problems.

In some embodiments, video capture includes retrieving video of opportunity, such as available in a large collection of videos, such as YouTube. In some embodiments, YouTube videos and videos registered with smart phones by parents were combined to study two infants longitudinally and seven infants cross-sectionally. The OpenPose software for pose estimation was used to produce the kinematic skeleton data in from the videos. Sample longitudinal tracking of an infant at 6 days after birth, at 5 weeks and at 17 weeks from this embodiment, are depicted in FIG. 10C through FIG. 10E.

In some embodiments, participants are included who are comfortable providing the requested information. The participants' pool is expanded to include infants who are deemed healthy, as well as those born pre-maturely, infants with family members who have or who themselves have known genetic issues which lead to neurodevelopmental disorders, and infants who had birth complications. This would provide a comparative model to infants who are on what is deemed a developmentally good trajectory, and allow better characterization, detection, and training for infants at-risk. Video capture operators, such as parents or other caregivers, are instructed to avoid clothing around the legs of the infant, so that the lower body can be included in the pose estimation analysis because the pose capture algorithm can operate more easily.

In such embodiments, more information from participants is requested than in previous embodiments. This includes any relevant health information for the infants and their parents. Additionally, more physical measurements are requested, including body weight, body height, and head circumference, at the time of each video submitted. In some embodiments clinical scores are retrieved from clinicians for the infants in the videos as a frame of reference for the current clinical metrics and milestones of development.

Reafferent feedback to the brain is involved to ensure development. These postures of FIG. 10F through FIG. 10J are indicative of another extremely important mechanism in development, “feedforward control” based on intent and error correction. This mechanism is critical to develop a predictive code and compensate for internal delays in sensory transduction and transmission across the motor systems. Understanding how these mechanisms mature in tandem with the emergence of theory of mind and other cognitive abilities help further bridge the principles of kinesthetic reafference, and the internal models of neuromotor control with principles of embodied cognition. This will be recognized while characterizing the emergence of anticipatory postural adjustments, many of which have been evaluated in these postures.

In some embodiments, the 2-dimensional joint trajectories which are acquired by video, are extended to 3-dimensional means by utilizing new models and validating such models with non-invasive physical wearable sensors. To advance data acquisition toward more advanced video-based methods, they are first validated and developed with physical measurements as ground truth. The methods presented here offer ways to validate such data and move the field toward new computational approaches to neurodevelopmental research.

In some embodiments, dyadic interactions between the infant and the caregiver are also of interest. In such embodiments, the motions of the caregiver in tandem with the infant's motions are analyzed together.

### 3. Hardware Overview

FIG. 11 is a block diagram that illustrates a computer system 1100 upon which an embodiment of the invention may be implemented. Computer system 1100 includes a communication mechanism such as a bus 1110 for passing information between other internal and external components of the computer system 1100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1100, or a portion thereof, constitutes a means for performing one or more steps of one or more methods described herein.

A sequence of binary digits constitutes digital data that is used to represent a number or code for a character. A bus 1110 includes many parallel conductors of information so that information is transferred quickly among devices coupled to the bus 1110. One or more processors 1102 for processing information are coupled with the bus 1110. A processor 1102 performs a set of operations on information. The set of operations include bringing information in from the bus 1110 and placing information on the bus 1110. The set of operations also typically include comparing two or more units of information, shifting positions of units of information, and combining two or more units of information, such as by addition or multiplication. A sequence of operations to be executed by the processor 1102 constitutes computer instructions.

Computer system 1100 also includes a memory 1104 coupled to bus 1110. The memory 1104, such as a random access memory (RAM) or other dynamic storage device, stores information including computer instructions. Dynamic memory allows information stored therein to be changed by the computer system 1100. RAM allows a unit of information stored at a location called a memory address to be stored and retrieved independently of information at neighboring addresses. The memory 1104 is also used by the processor 1102 to store temporary values during execution of computer instructions. The computer system 1100 also includes a read only memory (ROM) 1106 or other static storage device coupled to the bus 1110 for storing static information, including instructions, that is not changed by the computer system 1100. Also coupled to bus 1110 is a non-volatile (persistent) storage device 1108, such as a magnetic disk or optical disk, for storing information, including instructions, that persists even when the computer system 1100 is turned off or otherwise loses power.

Information, including instructions, is provided to the bus 1110 for use by the processor from an external input device 1112, such as a keyboard containing alphanumeric keys operated by a human user, or a sensor. A sensor detects conditions in its vicinity and transforms those detections into signals compatible with the signals used to represent information in computer system 1100. Other external devices coupled to bus 1110, used primarily for interacting with humans, include a display device 1114, such as a cathode ray tube (CRT) or a liquid crystal display (LCD), for presenting images, and a pointing device 1116, such as a mouse or a trackball or cursor direction keys, for controlling a position of a small cursor image presented on the display 1114 and issuing commands associated with graphical elements presented on the display 1114.

In the illustrated embodiment, special purpose hardware, such as an application specific integrated circuit (IC) 1120, is coupled to bus 1110. The special purpose hardware is configured to perform operations not performed by processor 1102 quickly enough for special purposes. Examples of application specific ICs include graphics accelerator cards for generating images for display 1114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1100 also includes one or more instances of a communications interface 1170 coupled to bus 1110. Communication interface 1170 provides a two-way communication coupling to a variety of external devices that operate with their own processors, such as printers, scanners and external disks. In general the coupling is with a network link 1178 that is connected to a local network 1180 to which a variety of external devices with their own processors are connected. For example, communication interface 1170 may be a parallel port or a serial port or a universal serial bus (USB) port on a personal computer. In some embodiments, communications interface 1170 is an integrated services digital network (ISDN) card or a digital subscriber line (DSL) card or a telephone modem that provides an information communication connection to a corresponding type of telephone line. In some embodiments, a communication interface 1170 is a cable modem that converts signals on bus 1110 into signals for a communication connection over a coaxial cable or into optical signals for a communication connection over a fiber optic cable. As another example, communications interface 1170 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1170 sends and receives electrical, acoustic or electromagnetic signals, including infrared and optical signals, that carry information streams, such as digital data.

The term computer-readable medium is used herein to refer to any medium that participates in providing information to processor 1102, including instructions for execution. Such a medium may take many forms, including, but not limited to, non-volatile media, volatile media and transmission media. Non-volatile media include, for example, optical or magnetic disks, such as storage device 1108. Volatile media include, for example, dynamic memory 1104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1102, except for carrier waves and other signals.

Logic encoded in one or more tangible media includes one or both of processor instructions on a computer-readable storage media and special purpose hardware, such as ASIC 1120.

Network link 1178 typically provides information communication through one or more networks to other devices that use or process the information. For example, network link 1178 may provide a connection through local network 1180 to a host computer 1182 or to equipment 1184 operated by an Internet Service Provider (ISP). ISP equipment 1184 in turn provides data communication services through the public, world-wide packet-switching communication network of networks now commonly referred to as the Internet 1190. A computer called a server 1192 connected to the Internet provides a service in response to information received over the Internet. For example, server 1192 provides information representing video data for presentation at display 1114.

The invention is related to the use of computer system 1100 for implementing the techniques described herein. According to one embodiment of the invention, those techniques are performed by computer system 1100 in response to processor 1102 executing one or more sequences of one or more instructions contained in memory 1104. Such instructions, also called software and program code, may be read into memory 1104 from another computer-readable medium such as storage device 1108. Execution of the sequences of instructions contained in memory 1104 causes processor 1102 to perform the method steps described herein. In alternative embodiments, hardware, such as application specific integrated circuit 1120, may be used in place of or in combination with software to implement the invention. Thus, embodiments of the invention are not limited to any specific combination of hardware and software.

The signals transmitted over network link 1178 and other networks through communications interface 1170, carry information to and from computer system 1100. Computer system 1100 can send and receive information, including program code, through the networks 1180, 1190 among others, through network link 1178 and communications interface 1170. In an example using the Internet 1190, a server 1192 transmits program code for a particular application, requested by a message sent from computer 1100, through Internet 1190, ISP equipment 1184, local network 1180 and communications interface 1170. The received code may be executed by processor 1102 as it is received, or may be stored in storage device 1108 or other non-volatile storage for later execution, or both. In this manner, computer system 1100 may obtain application program code in the form of a signal on a carrier wave.

Various forms of computer readable media may be involved in carrying one or more sequence of instructions or data or both to processor 1102 for execution. For example, instructions and data may initially be carried on a magnetic disk of a remote computer such as host 1182. The remote computer loads the instructions and data into its dynamic memory and sends the instructions and data over a telephone line using a modem. A modem local to the computer system 1100 receives the instructions and data on a telephone line and uses an infra-red transmitter to convert the instructions and data to a signal on an infra-red a carrier wave serving as the network link 1178. An infrared detector serving as communications interface 1170 receives the instructions and data carried in the infrared signal and places information representing the instructions and data onto bus 1110. Bus 1110 carries the information to memory 1104 from which processor 1102 retrieves and executes the instructions using some of the data sent with the instructions. The instructions and data received in memory 1104 may optionally be stored on storage device 1108, either before or after execution by the processor 1102.

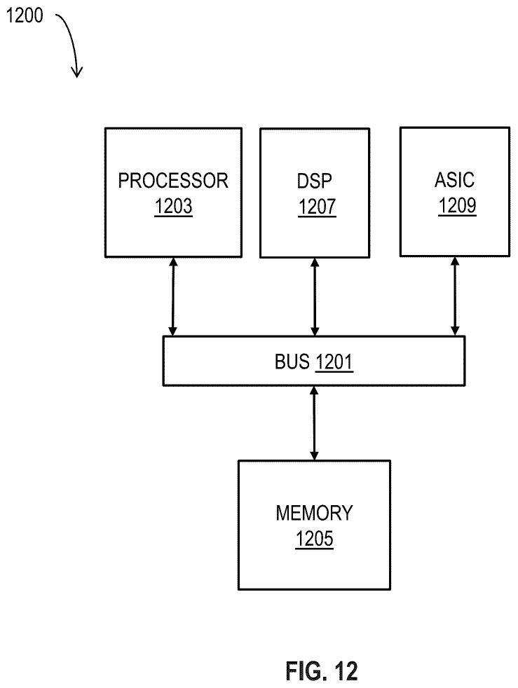

FIG. 12 illustrates a chip set 1200 upon which an embodiment of the invention may be implemented. Chip set 1200 is programmed to perform one or more steps of a method described herein and includes, for instance, the processor and memory components described with respect to FIG. 11 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1200, or a portion thereof, constitutes a means for performing one or more steps of a method described herein.

In one embodiment, the chip set 1200 includes a communication mechanism such as a bus 1201 for passing information among the components of the chip set 1200. A processor 1203 has connectivity to the bus 1201 to execute instructions and process information stored in, for example, a memory 1205. The processor 1203 may include one or more processing cores with each core configured to perform independently. A multi-core processor enables multiprocessing within a single physical package. Examples of a multi-core processor include two, four, eight, or greater numbers of processing cores. Alternatively or in addition, the processor 1203 may include one or more microprocessors configured in tandem via the bus 1201 to enable independent execution of instructions, pipelining, and multithreading. The processor 1203 may also be accompanied with one or more specialized components to perform certain processing functions and tasks such as one or more digital signal processors (DSP) 1207, or one or more application-specific integrated circuits (ASIC) 1209. A DSP 1207 typically is configured to process real-world signals (e.g., sound) in real time independently of the processor 1203. Similarly, an ASIC 1209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1203 and accompanying components have connectivity to the memory 1205 via the bus 1201. The memory 1205 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1205 also stores the data associated with or generated by the execution of one or more steps of the methods described herein.

FIG. 13 is a diagram of exemplary components of a mobile terminal 1300 (e.g., cell phone handset) for communications, which is capable of operating in the system of FIG. 1A, according to one embodiment. In some embodiments, mobile terminal 1301, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1303, a Digital Signal Processor (DSP) 1305, and a receiver/transmitter unit including a microphone gain control unit and a speaker gain control unit. A main display unit 1307 provides a display to the user in support of various applications and mobile terminal functions that perform or support the steps as described herein. The display 1307 includes display circuitry configured to display at least a portion of a user interface of the mobile terminal (e.g., mobile telephone). Additionally, the display 1307 and display circuitry are configured to facilitate user control of at least some functions of the mobile terminal. An audio function circuitry 1309 includes a microphone 1311 and microphone amplifier that amplifies the speech signal output from the microphone 1311. The amplified speech signal output from the microphone 1311 is fed to a coder/decoder (CODEC) 1313.

A radio section 1315 amplifies power and converts frequency in order to communicate with a base station, which is included in a mobile communication system, via antenna 1317. The power amplifier (PA) 1319 and the transmitter/modulation circuitry are operationally responsive to the MCU 1303, with an output from the PA 1319 coupled to the duplexer 1321 or circulator or antenna switch, as known in the art. The PA 1319 also couples to a battery interface and power control unit 1320.

In use, a user of mobile terminal 1301 speaks into the microphone 1311 and his or her voice along with any detected background noise is converted into an analog voltage. The analog voltage is then converted into a digital signal through the Analog to Digital Converter (ADC) 1323. The control unit 1303 routes the digital signal into the DSP 1305 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1325 for compensation of any frequency-dependent impairments that occur during transmission though the air such as phase and amplitude distortion. After equalizing the bit stream, the modulator 1327 combines the signal with a RF signal generated in the RF interface 1329. The modulator 1327 generates a sine wave by way of frequency or phase modulation. In order to prepare the signal for transmission, an up-converter 1331 combines the sine wave output from the modulator 1327 with another sine wave generated by a synthesizer 1333 to achieve the desired frequency of transmission. The signal is then sent through a PA 1319 to increase the signal to an appropriate power level. In practical systems, the PA 1319 acts as a variable gain amplifier whose gain is controlled by the DSP 1305 from information received from a network base station. The signal is then filtered within the duplexer 1321 and optionally sent to an antenna coupler 1335 to match impedances to provide maximum power transfer. Finally, the signal is transmitted via antenna 1317 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1301 are received via antenna 1317 and immediately amplified by a low noise amplifier (LNA) 1337. A down-converter 1339 lowers the carrier frequency while the demodulator 1341 strips away the RF leaving only a digital bit stream. The signal then goes through the equalizer 1325 and is processed by the DSP 1305. A Digital to Analog Converter (DAC) 1343 converts the signal and the resulting output is transmitted to the user through the speaker 1345, all under control of a Main Control Unit (MCU) 1303 which can be implemented as a Central Processing Unit (CPU) (not shown).

The MCU 1303 receives various signals including input signals from the keyboard 1347. The keyboard 1347 and/or the MCU 1303 in combination with other user input components (e.g., the microphone 1311) comprise a user interface circuitry for managing user input. The MCU 1303 runs a user interface software to facilitate user control of at least some functions of the mobile terminal 1301 as described herein. The MCU 1303 also delivers a display command and a switch command to the display 1307 and to the speech output switching controller, respectively. Further, the MCU 1303 exchanges information with the DSP 1305 and can access an optionally incorporated SIM card 1349 and a memory 1351. In addition, the MCU 1303 executes various control functions required of the terminal. The DSP 1305 may, depending upon the implementation, perform any of a variety of conventional digital processing functions on the voice signals. Additionally, DSP 1305 determines the background noise level of the local environment from the signals detected by microphone 1311 and sets the gain of microphone 1311 to a level selected to compensate for the natural tendency of the user of the mobile terminal 1301.

The CODEC 1313 includes the ADC 1323 and DAC 1343. The memory 1351 stores various data including call incoming tone data and is capable of storing other data including music data received via, e.g., the global Internet. The software module could reside in RAM memory, flash memory, registers, or any other form of writable storage medium known in the art. The memory device 1351 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1349 carries, for instance, important information, such as the cellular phone number, the carrier supplying service, subscription details, and security information. The SIM card 1349 serves primarily to identify the mobile terminal 1301 on a radio network. The card 1349 also contains a memory for storing a personal telephone number registry, text messages, and user specific mobile terminal settings.

In some embodiments, the mobile terminal 1301 includes a digital camera comprising an array of optical detectors, such as charge coupled device (CCD) array 1365. The output of the array is image data that is transferred to the MCU for further processing or storage in the memory 1351 or both. In the illustrated embodiment, the light impinges on the optical array through a lens 1363, such as a pin-hole lens or a material lens made of an optical grade glass or plastic material. In the illustrated embodiment, the mobile terminal 1301 includes a light source 1361, such as a LED to illuminate a subject for capture by the optical array, e.g., CCD 1365. The light source is powered by the battery interface and power control module 1320 and controlled by the MCU 1303 based on instructions stored or loaded into the MCU 1303.

### 4. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 5. REFERENCES

The following articles are hereby incorporated by reference as if fully set forth herein, except for terminology inconsistent with that used herein.
Cao, Z.; Hidalgo Martinez, G.; Simon, T.; Wei, S. E.; Sheikh, Y. A. OpenPose: Realtime Multi-Person 2D Pose Estimation Using Part Affinity Fields. IEEE Trans. Pattern Anal. Mach. Intell. 2021, 43, 172-186.
Cao, Z.; Simon, T.; Wei, S. E.; Sheikh, Y. Realtime Multi-Person 2D Pose Estimation Using Part Affinity Fields. In Proceedings of the 30th IEEE Conference on Computer Vision and Pattern Recognition, Honolulu, HI, USA, 21-26 Jul. 2017; pp. 1302-1310
Fang, H.; Xie, S.; Lu, C. RMPE: Regional Multi-Person Pose Estimation. arXiv 2018, arXiv: 1612.001737
Insafutdinov, E.; Pishchulin, L.; Andres, B.; Andriluka, M.; Schiele, B. Deepercut: A Deeper, Stronger, and Faster Multi-Person Pose Estimation Model. In Computer Vision—ECCV 2016; Lecture Notes in Computer Science (Including Subseries Lecture Notes in Artificial Intelligence and Lecture Notes in Bioinformatics); Springer International Publishing: Cham, Switzerland, 2016; pp. 34-50.
Insafutdinov, E.; Andriluka, M.; Pishchulin, L.; Tang, S.; Levinkov, E.; Andres, B.; Schiele, B. ArtTrack: Articulated Multi-Person Tracking in the Wild. In Proceedings of the 30th IEEE Conference on Computer Vision and Pattern Recognition, Honolulu, HI, USA, 21-26 Jul. 2017; pp. 6457-6465
Mathis, A.; Mamidanna, P.; Cury, K. M.; Abe, T.; Murthy, V. N.; Mathis, M. W.; Bethge, M. DeepLabCut: Markerless Pose Estimation of User-Defined Body Parts with Deep Learning. Nat. Neurosci. 2018, 21, 1281-1289.
Stenum, J: Cherry-Allen, K. M.; Pyles, C. O.; Reetzke, R. D.; Vignos, M. F; Ryan T. Roemmich, R. T.; Applications of Pose Estimation in Human Health and Performance across the Lifespan, Sensors 2021, 21, p 7315.
Toshev, A.; Szegedy, C. DeepPose: Human Pose Estimation via Deep Neural Networks. In Proceedings of the IEEE Computer Society Conference on Computer Vision and Pattern Recognition, Columbus, OH, USA, 23-28 Jun. 2014; pp. 1653-1660.
Fang, H.; Xie, S.; Lu, C. RMPE: Regional Multi-Person Pose Estimation. arXiv 2018, arXiv: 1612.001737

What is claimed is:

1. A method for determining a condition of a subject, the method comprising:
recording video data of a subject;
determining a micromovements profile of each of one or more anatomical locations on the subject using pose algorithms on the recorded video data;
determining a first empirical distribution of micromovement spikes in each micromovement profile, and
determining a condition of the subject based on a distance of the first empirical distribution from a predetermined distribution of micromovement spikes in a population of control individuals.

2. The method as recited in claim 1, wherein the population of control individuals includes a plurality of populations of control individuals, each population having a different predetermined distribution of micromovement spikes and each population associated with a different neurodevelopmental condition.

3. The method as recited in claim 1, wherein said recording video data further comprising recording video data of a subject before or after, or both, an auditory signal is delivered to the subject during an auditory brainstem response (ABR) test.

4. The method as recited in claim 3, wherein the predetermined distribution of micromovement spikes in the population of control individuals is associated with a predetermined distribution of micropeaks in ABR test data for the population of control individuals and the predetermined distribution of micropeaks in ABR test data is associated with a particular neurodevelopmental condition.

5. The method as recited in claim 4, where the population of control individuals includes a plurality of populations of control individuals, each population having a different predetermined distribution of micropeaks in ABR test data, and each population is associated with a different neurodevelopmental condition.

6. The method as recited in claim 2, where the plurality of populations of control individuals, includes a first population associated with normal development and different second population associated with autism spectrum disorder (ASD).

7. The method as recited in claim 1, wherein the first empirical distribution of micromovement spikes is an empirical distribution of a prominence for each micromovement spike.

8. The method as recited in claim 1, wherein the first empirical distribution of micromovement spikes is an empirical distribution of a width for each micromovement spike:

9. The method as recited in claim 1, wherein the first empirical distribution of micromovement spikes is an empirical distribution of an amplitude for each micromovement spike.

10. The method as recited in claim 1, wherein the first empirical distribution of micromovement spikes is an empirical distribution of a relative latency for each micromovement spike.

11. The method as recited in claim 1, wherein the distance is an Earthmover's distance between distributions.

12. The method as recited in claim 1, wherein the first empirical distribution is described by a plurality of distribution parameters.

13. The method as recited in claim 12, wherein the distribution parameters include mean, variance and skewness.

14. The method as recited in claim 12, wherein the distribution parameters include shape and scale of a Gamma Function.
